(19) United States
(12) Patent Application Publication
(10) Pub. No.: US 2023/0398237 A1
(43) Pub. Date: Dec. 14, 2023

(54) COMPOSITION COMPRISING POLYETHYLENE GLYCOL CONJUGATED TO INDOCYANINE GREEN AND METHODS OF USE

(71) Applicant: BOARD OF REGENTS, THE UNIVERSITY OF TEXAS SYSTEM, Austin, TX (US)

(72) Inventors: Jie ZHENG, Plano, TX (US); Bujie DU, Richardson, TX (US); Mengxiao YU, Frisco, TX (US)

(21) Appl. No.: 18/022,670

(22) PCT Filed: Aug. 24, 2021
(86) PCT No.: PCT/US21/47248

§ 371 Date: Feb. 22, 2023

Related U.S. Application Data
(60) Provisional application No. 63/069,161, filed on Aug. 24, 2020.

(51) Int. Cl. A61K 49/00; A61B 5/20

(52) U.S. Cl. CPC A61K 49/0034; A61B 5/201; A61K 49/0054

(57) ABSTRACT

The present disclosure provides polyethylene glycol-indocyanine green conjugates as well as the methods of using such conjugates, such as diagnosis, tissue imaging, temporal monitoring, and treatment.

### RELATED APPLICATIONS

This application claims the benefit of U.S. Provisional Application No. 63/069,161, filed Aug. 24, 2020, the contents of which are incorporated herein by reference in their entireties.

### GOVERNMENT SUPPORT

This invention was made with government support under Grant No. R01 DK103363 and Grant No. R01 DK115986 awarded by the National Institutes of Health. The government has certain rights in the invention.

### BACKGROUND

The kidneys are a major organ for rapid removal of endogenous wastes and exogenous drugs/toxins from the body. However, the exact elimination pathway for a solute strongly depends on its interactions with kidney compartments. Upon the interactions with kidney compartments, molecules can take two different pathways, glomerular filtration and renal tubular secretion, to be eliminated through the kidneys. For some molecules that have little interactions with kidney compartments and are smaller than 6 nm in hydrodynamic diameter (or lower than 40 kDa in molecular weight), they can be rapidly and passively eliminated through the glomerular filtration membrane. On the other hand, some other molecules can be actively excreted from peritubular capillaries into the lumen of the proximal tubules by binding the transporters on the basolateral side of proximal tubular cells, influx into the cells and efflux from the luminal side. The newly developed renal clearable nanofluorophores and organic dyes are mainly taking the glomerular filtration pathway and have been used for detecting kidney dysfunction or improving positive contrast of many cancers for fluorescence-guided surgery. However, none of them was reported to selectively target primary kidney cancers over normal kidney tissues and visualize the tumor margins with positive contrast (hyperfluorescence), which is highly demanded in fluorescence-guided partial nephrectomy to preserve kidney function and improve the quality of life of patients with kidney cancer. In addition, due to high blood perfusion of normal renal parenchyma, it remains challenging to selectively deliver therapeutic agents (such as agent for photothermal therapy, photodynamic therapy, chemotherapy, immunotherapy and radiation therapy) and medical imaging agents (such as agents for photoacoustic imaging, computed tomography, positron emission tomography, single-photon emission computerized tomography, and magnetic resonance imaging) into the kidney cancer cells at a higher concentration than nearby normal kidney tissues. Considering that over 90 percent of kidney cancer is renal cell carcinoma (RCC) originating from the renal tubular epithelial cell, the fundamental understanding of the interactions and transport of renal clearable dyes and nanoparticles with/in renal tubules is essential to designing new strategies for selectively targeting of RCC.

While proximal tubular secretion plays an important role in the rapid removal of endogenous substances and exogenous drugs or toxins, proximal tubules are also very vulnerable to endogenous cytokines and exogenous drugs or toxins, resulting in the impairment in tubular secretion function, kidney injury, and even kidney failure. Compared to glomerular filtration function that can be readily estimated with either endogenous serum creatinine or exogenous markers such as radiolabeling tracers or fluorescent inulin, tubular secretion function can only be quantified using few exogenous markers so far. In the clinics, exogenous functional markers such as para-aminohippurate (PAH) are intravenously infused into the patients. By analyzing their blood and urine concentrations with “off-line” colorimetric or chromatographic methods, clinicians can quantify remaining tubular secretion function and personalize treatment plans to minimize the potential side effects and nephrotoxicity. However, the “off-line” analysis is time-consuming and fails to address the urgent clinical needs in acute kidney injuries. To address this challenge, radionuclides such as ⁹⁹ mTc-mercaptoacetyltriglycine (MAG3) are being introduced for real-time monitoring of tubular secretion function; but potential radiation hazards and sophisticate clinical settings preclude them from being used in family clinics as well as in rural areas with limited medical resources. With the emergence of portable or wearable optoelectronics, it is highly desirable to develop exogenous nonradiative optical markers for remote assessment of proximal tubular secretion function and its impairment at the early stage; so that prognostic planning and the early treatment can be timely made for patients in the remote areas with limited medical sources to prevent kidney failure.

### SUMMARY

In one aspect, the present disclosure provides methods of diagnosing a disease or condition associated with abnormal expression of an influx or efflux transporter in a subject, comprising:
administering to the subject a composition comprising polyethylene glycol (PEG) conjugated to indocyanine green (ICG) (“ICG-PEG conjugate”);
determining a concentration of the ICG-PEG conjugate in a biological sample obtained from the subject;
comparing the concentration of the ICG-PEG conjugate with a reference level; and
determining that the subject has the disease or condition if the concentration of the ICG-PEG conjugate is significantly greater or lower than the reference level.

In another aspect, the present disclosure further provides methods of diagnosing a disease or condition associated with abnormal expression of an influx or efflux transporter in a subject, comprising:
administering to the subject a composition comprising an ICG-PEG conjugate;
measuring an intensity of a signal from the ICG-PEG conjugate in a tissue of the subject; comparing the intensity with a reference level; and
determining that the subject has the disease or condition if the intensity is significantly greater or lower than the reference level.

In another aspect, the present disclosure provides methods of monitoring kidney secretion function of a subject, comprising:
administering to the subject a composition comprising an ICG-PEG conjugate;
determining a first concentration of the ICG-PEG conjugate in a first biological sample obtained from the subject at a first time point;
determining a second concentration of the ICG-PEG conjugate in a second biological sample obtained from the subject at a second time point, wherein the second time point is after the first time point;
determining renal clearance kinetics based on the first concentration and the second concentration; and
optionally comparing the renal clearance kinetics with a reference level.

In another aspect, the present disclosure provides a method of monitoring kidney secretion function of a subject, comprising:
administering to the subject a composition comprising an ICG-PEG conjugate;
measuring, at a first time point, a first intensity of a signal from the ICG-PEG conjugate in a tissue of the subject; and
measuring, at a second time point, a second intensity of a signal from the ICG-PEG conjugate in the tissue of the subject.

In another aspect, the present disclosure provides methods of treating a disease or condition associated with abnormal expression of an influx or efflux transporter in a subject in need thereof, comprising administering to the subject a composition comprising an ICG-PEG conjugate.

In another aspect, the present disclosure provides a method of measuring an expression level of an influx or efflux transporter in a subject, comprising:
administering to the subject a composition comprising an ICG-PEG conjugate; determining a concentration of the ICG-PEG conjugate in a biological sample obtained from the subject; and
determining the expression level of the influx or efflux transporter based on the concentration of the ICG-PEG conjugate.

In another aspect, the present disclosure provides a method of measuring an expression level of an influx or efflux transporter in a subject, comprising:
administering to the subject a composition comprising an ICG-PEG conjugate;
measuring an intensity of a signal from the ICG-PEG conjugate in a tissue of the subject; and
determining the expression level of the influx or efflux transporter based on the intensity.

In another aspect, the present disclosure provides methods of detecting a liver disease in a subject, comprising:
administering to the subject a composition comprising an ICG-PEG conjugate, wherein the PEG has a molecular weight of at least 100 Da to less than 2 kDa;
determining a concentration of the ICG-PEG conjugate in a urine sample obtained from the subject;
comparing the concentration of the ICG-PEG conjugate with a reference level; and
determining that the subject has the liver disease when the concentration of the ICG-PEG conjugate is significantly less than the reference level.

### BRIEF DESCRIPTION OF THE DRAWINGS

FIG. 1A is a figure showing the comparison between ICG and ICG-PEG45 in chemical structures, molecular weight (MW), net charge, partition coefficient (logD), and serum protein binding.

FIG. 1B is an fluorescence image showing in vivo imaging of mice intravenously injection of ICG and ICG-PEG45 at 10 min post injection and ex vivo images of harvested liver (Li), kidneys (Kid), heart (He), spleen (Sp) and urine collected from bladder at 10 min post intravenous injections (Ex/Em filters: 790/830 nm). The mice were placed in prone position to collect signals from the liver.

FIG. 1C is a bar graph showing clearance percentage of ICG and ICG-PEG45 in urine and feces, respectively, at 24 h post intravenous injection.

FIG. 1D is a graph showing clearance percentage of ICG, ICG-PEG22, ICG-PEG45 and ICG-PEG220 in feces and urine at 24 h post injections. n=3. PEG45 behaved as a turning point in terms of renal clearance of ICG-PEGn.

FIG. 1E is a set of fluorescent image and a graph showing real-time noninvasive kidney imaging before and after intravenous of injection of ICG-PEG45 (Ex/Em filters: 790/830 nm) (i) and time-fluorescence intensity curves of two kidneys within 30 min post injection of ICG-PEG45 (ii). To make a comparison, the fluorescent kidney kinetics curve of 800CW-PEG45 was presented in the dotted line. To collect the signals of the kidneys, the mouse was placed in supine position on the imaging stage.

FIG. 1F is a set of fluorescence images of glomerulus and tubules at tissue level at 5 min and 1 h post injection of 800CW-PEG45 and ICG-PEG45. G, glomerulus, PT, proximal tubule. Kidney tissue was stained by Hematoxylin and Eosin (H&E). Fluorescence images were taken at 775/845 nm for ICG-PEG45 and 720/790 nm for 800CW-PEG45. Scalar bar is 20 μm.

FIG. 1G is a set of bar graphs showing the renal clearance efficiencies of ICG-PEG45 (i) and 800CW-PEG45 (ii) at 30 min post injection under control condition and probenecid-treated condition. n=3. *represents statistically different based on student t-test, P<0.05. N. S. represents no significant difference based on student-test, P>0.05.

FIG. 1H is set of schematic diagrams of the clearance alteration of ICG after conjugation of PEG45.

FIG. 2A is a schematic diagram of an established mouse model primary, orthotopic renal cell carcinoma (RCC).

FIG. 2B is a set of fluorescent images showing real-time noninvasive kidney imaging of orthotopic papillary RCC implanted mice after intravenous injection of ICG-PEG45 (Ex/Em filters: 790/830 nm). BF, bright field. Left kidney of mice was implanted with RCC (LK: w/tumor) and right kidney was normal kept (RK: w/o tumor). The mouse was placed in supine position on the imaging stage.

FIG. 2C is a plot showing time-fluorescence intensity of two kidneys locations and background skin within 24 h post injection of ICG-PEG45. RS, right background skin. LS, left background skin.

FIG. 2D is a graph showing clearance percentage of at 24 h post injection of ICG-PEG45 of RK (w/o T) and LK (w/T). The value was defined as [peak value-intensity at 24 h]/peak value]×100%. n=3, p value was calculated based on student t-test.

FIG. 2E is set of ex vivo images of cancerous kidney with papillary RCC (LK) and contralateral kidney without RCC (RK) at 24 h post intravenous injection of ICG-PEG45.

FIG. 2F is a set of fluorescent images showing the distribution of ICG-PEG45 in papillary RCC tissue at 24 h post injection.

FIG. 2G is a set of ex vivo images of cancerous kidney with patient-derived xenograft (PDX) clear cell RCC (ccRCC) and contralateral kidney without RCC at 68 h post injection of ICG-PEG45 and fluorescence imaging of tissue level after H&E staining. BF, bright field. Fl, fluorescence from ICG-PEG45. The dot line represents the margin between normal kidney tissue and ccRCC.

FIG. 2H is a graph showing the comparison of RCC contrast index among ICG-PEG45, ICG and 800CW-PEG45. Hypo, hypofluorescent (intensity of normal kidneys>intensity of kidney cancer). Iso, isofluorescent (intensity of normal kidneys=intensity of kidney cancer). Hyper, hyperfluorescent (intensity of normal kidneys<intensity of kidney cancer).

FIG. 2I is a table showing the correlation of clearance pathways with RCC targeting by summarizing investigated probes.

FIG. 3A is set of images showing the P-glycoprotein (P-gP) expression level on the membrane of normal kidney proximal tubular cell (HK2) and renal cell carcinoma cell (529B) as well as at the tissue level.

FIG. 3B is a set of fluorescent images showing the cellular uptake fluorescence imaging of ICG-PEG45 in HK2 and 529B before (CSA−) and after (CSA+) inhibition of P-gP-mediated efflux by the cyclosporin A (CSA) treatment. scalar bar is 10 μm. BF, bright field. Fl, fluorescence.

FIG. 3C is a bar graph showing the quantification of intracellular fluorescence intensity of ICG-PEG45. *** represents statistically different based on student t-test and P<0.0005. N.S. represents no significant difference based on student-test, P>0.05.

FIG. 3D is a schematic diagram of distinct cellular efflux of ICG-PEG45 in normal kidney proximal tubular cell and RCC cell, which is the origin of RCC hyperfluorescent contrast by ICG-PEG45.

FIG. 4A is a scheme showing that RCC could migrate to brain, bone and lung.

FIG. 4B is a set of in vivo noninvasive bioluminescence images and fluorescence images of mice bearing RCC metastatic tumors at 24 h after injection ICG-PEG45. BLI, bioluminescence images. Fl, fluorescence. R, right, L, left.

FIG. 4C is a set of ex vivo images of tumor near spine and brain at 24 h post injection of ICG-PEG45. BLI, bioluminescence images. Fl, fluorescence.

FIG. 4D is a set of ex vivo images of upper limbs and lower limbs at 24 h post injection of ICG-PEG45. BLI, bioluminescence images. Fl, fluorescence.

FIG. 4E is a set of in vivo noninvasive images of other mice bearing RCC metastatic tumors at 24 h post injection of ICG-PEG45 (left) and ex vivo FL images of lower limbs (with and without muscle) and color images (without muscle) (right).

FIG. 4F is a photo as well as H&E pathology images showing 24 h post injection of ICG-PEG45. The images of upper limbs were in FIG. 29.

FIG. 5A is graph shwong the creatinine levels in the normal and diseased kidney with tubular injury induced by 10 mg/kg of cisplatin.

FIG. 5B is a graph showing the PAH levels in the normal and diseased kidney with tubular injury induced by 10 mg/kg of cisplatin.

FIG. 5C is a tunnel TUNEL assay image that confirms the tubular injury at 10 mg/kg cisplatin dose.

FIG. 5D is a KIM-1 immunostaining image that confirms the tubular injury at 10 mg/kg cisplatin dose.

FIG. 6 is a graph showing therenal clearance efficiency of ICG-PEG45 in normal and diseased mice induced by cisplatin at the doses of 10 mg/kg and 20 mg/kg, respectively. The significant reduction in the renal clearance was observed.

FIG. 7A is a noninvasive in vivo image of a mouse with orthotopic RCC xenograft on the left kidney at 24 h post intravenous injection of ICG-PE45-DOTA (200 μL, 40 μM). The left kidney of the mouse was implanted with papillary RCC cell line (ACHN) transfected with luciferase-expression vector, and right kidney was normal kept. At 10 min after intraperitoneal injection of luciferase substrate, strong bioluminescence signal was detected on the left kidney, indicating the growth of RCC. Bioluminescence image was overlapped with brightfield image to show the location of the bioluminescence signal on the left kidney.

FIG. 7B is a near-infrared fluorescence image of the same mouse suggests that ICG-PE45-DOTA can specifically accumulate in the left kidney with primary RCC but can be cleared from the normal right kidney (Ex/Em filters: 760/845 nm).

FIG. 8A is a bioluminescence image of the left and right kidneys that were cut in half longitudinally.

FIG. 8B a photo of the kidneys shown in FIG. 8A.

FIG. 8C is a near-infrared fluorescence image of the kidneys shown in FIG. 8A. The fluorescent signal indicated that ICG-PE45-DOTA can specifically target the malignant kidney tissue (Ex/Em filters: 760/845 nm).

FIG. 9 is a set of real-time in vivo images of mice bearing MCF-7 tumor after injection of ICG and ICG-PEG45, respectively. Ex/Em: 790/830 nm. Tumor sites were pointed out by arrow or triangle.

FIG. 10A is a set of images showing the location of ICG and ICG-PEG45 in MCF-7 cells after 4 h incubation. Nuclei were stained by Hoechst. BF, bright field. Scale bar is 20 nm.

FIG. 10B is a bar graph showing MCF-7 cellular uptake efficiencies of ICG and ICG-PEG45 with incubation time of 12 h. The conjugation of PEG45 to ICG reduced its cellular uptake efficiency for ˜10 times.

FIG. 11A is a set of real-time in vivo images of mice bearing triple-negative 4T1 tumor after injection of ICG-PEG45. BF, bright field. Tumor sites were pointed out by arrow or triangle.

FIG. 11B is graph showing tumor contrast index of ICG-PEG45 in mice bearing 4T1 tumors. Tumor contrast index was calculated by the ratio of intensity from tumor site over that from background tissue. The dotted line represents contrast index threshold (CI=2.5). The CI of ICG-PEG45 reached 2.5 at 3 h.

FIG. 11C is a time-fluorescence curves of ICG-PEG45 in tumors site (4T1) and background tissue. Decay half-lives of ICG-PEG45 in tumor sites and background tissue sites were calculated. T, tumor site, B, background tissue.

FIG. 11D shows the distribution of ICG-PEG45 in tumor tissue at 24 h post injection. Tumor tissue was H&E stained. Fluorescence images were taken at EX775/EM845 nm. Scalar bar is 20 μm.

FIGS. 12A and 12B are Hematoxylin and Eosin (H&E)-stained kidney section shows the local tubular injury caused by a surgical cut is in the renal cortex. The dilated renal tubules and interstitial infiltration of immune cells are labeled by stars.

FIGS. 12C and 12D are near-infrared fluorescence imaging of the distribution of ICG-PEG (MW of PEG=5000) in the kidney section. Red signal, ICG fluorescence. The cell nuclei were stained in blue by DAPI.

FIG. 13 is a scheme showing the synthesis of ICG-PEG45.

FIG. 14 is a set of UV-vis absorption and fluorescence spectra of ICG and ICG-PEG45.

FIG. 15 shows the serum protein binding test by agarose gel electrophoresis. The serum protein binding test of ICG and ICG-PEG45 was conducted by agarose gel electrophoresis in 10% fetal bovine serum (FBS) under 37° C. water bath for 30 min. Coomassie brilliant blue 250 (CBB) was used to stain FBS. Color pictures of CBB-stained protein were inserted. ICG completely binds protein, while conjugation of PEG45 molecules reduces its protein binding affinity.

FIG. 16 is a set of UV-vis absorption and fluorescence spectra of ICG-PEG22 and ICG-PEG220.

FIG. 17 is a set of real time in vivo images of ICG-PEG22 and ICG-PEG220 within 10 min post injection and ex vivo images of harvested organs. liver (Li), kidneys (Kid), heart (He), spleen (Sp) and urine collected from bladder at 10 min post intravenous injections (Ex/Em filters: 790/830 nm).

FIG. 18A shows the serum protein binding test by agarose gel electrophoresis of ICG-PEG22 and ICG-PEG220 in 10% fetal bovine serum (FBS) under 37° C. water bath for 30 min. Coomassie brilliant blue 250 (CBB) was used to stain FBS. Color pictures of CBB-stained protein were inserted.

FIG. 18B is a set of images showing the fluorescence intensity of ICG, ICG-PEG22, ICG-PEG45 and ICG-PEG220 in water and in 100% fetal bovine serum (FBS) (same dye concentration).

FIG. 18C is a curve showing the ratio of sum intensity in FBS over that in water of ICG, ICG-PEG22, ICG-PEG45 and ICG-PEG220. The ratio decreased with the increase of molecular weight of PEG, indicating that the protein binding of ICG is gradually decreasing with the increase of molecular weight of conjugated PEG molecules.

FIG. 19A is a set of bright field (BF) and fluorescence images at 1 min, 10 min and 30 min post intravenous injection of 4 μM ICG-PEG45. Kidney imaging of ICG-PEG45 with injection dosage of 4 μM. The concentration is 10 times lower than the dose used for ICG-PEG45 kidney imaging in the main text. The skin on the back side was removed to clearly monitor the fluorescence signals from kidneys.

FIG. 19B is a graph showing the kinetics of right kidney and left kidney over time. LK, left kidney. RK, right kidney. Kidney imaging of ICG-PEG45 with injection dosage of 4 μM. The concentration is 10 times lower than the dose used for ICG-PEG45 kidney imaging in the main text. The skin on the back side was removed to clearly monitor the fluorescence signals from kidneys.

FIG. 20A is a set of real time non-invasive kidney images post injection of ICG-PEG45.

FIG. 20B is a graph showing the kinetics curves of right kidney (RK), left kidney (LK) and skin over time.

FIG. 21 is a set of images showing the distribution of ICG-PEG45 in glomerulus and tubules at 10 min post intravenously injection (p.i.). BF, bright field. Fl, fluorescence. Scale bar is 20 μm. G, glomerulus, PT, proximal tubule, L, lumen.

FIG. 22A is a graph showing the signal intensity of ICG-PEG45 in peritubular capillary, tubular lumen and glomerulus at 5 min post injection.

FIG. 22B is a graph showing the signal intensity of ICG-PEG45 in peritubular capillary, tubular lumen and glomerulus at 1 h post injection. The p value is calculated based on student t-test.

FIG. 23 is a bar graph showing the effect of probenecid on the renal clearance efficiency of FITC-inulin. n=3. N.S. means no significant difference based on Student-t test, P>0.05.

FIG. 24A is a set of ex vivo fluorescent images of kidneys with RCC and without RCC after injection of ICG, at 24 h. BF, bright filed. Fl, fluorescence. RCC, renal cell carcinoma.

FIG. 24B is a set of ex vivo fluorescent images of kidneys with RCC and without RCC after injection of 800CW-PEG45, at 24 h. BF, bright filed. Fl, fluorescence. RCC, renal cell carcinoma.

FIG. 25 is a set of ex vivo RCC at 24 h post intravenous injection of ICG-Au25. RCC, renal cell carcinoma. BF, bright field. Fl, fluorescence.

FIG. 26A is a set of cellular uptake fluorescence images of ICG-PEG45 in HK2 and 529B before and after the tariquidar treatment. scalar bar is 20 μm. BF, bright field. Fl, fluorescence.

FIG. 26B is a bar graph showing the quantification of fluorescence intensity of ICG-PEG45. *** represents statistically different based on student t-test and P<0.0005. N.S. represents no significant difference based on student-test, P>0.05.

FIG. 27 shows the contrast index of joints with tumor and normal joints of mice after injection of ICG-PEG45 at 24 h. The contrast index (CI) is calculated by the intensity of part 1 over part 2 (circled in figures).

FIG. 28 is a set of ex vivo fluorescent images of renal cell carcinoma metastasis in lung by ICG-PEG45 at 24 h post intravenous injection.

FIG. 29 is a set of ex vivo images and color images of tumor-bearing upper limbs from mouse at 24 h post injection of ICG-PEG45 and H&E pathology images.

FIG. 30A is a graph showing the BUN levels at 4 days after cisplatin/saline treatment.

FIG. 30B is a graph showing the serum creatinine levels at 4 days after cisplatin/saline treatment.

FIG. 30C is a graph showing urinary KIM-1 and creatinine ratios at 4 days after cisplatin/saline treatment.

FIG. 30D is a set of images of immunofluorescence-stained renal tissues confirming the upregulated KIM-1 expression on 10 mg/kg and 20 mg/kg cisplatin-treated mouse kidneys.

FIG. 30E is a set of images showing terminal deoxynucleotidyl transferase dUTP nick end labeling (TUNEL) stained renal tissues confirmed the presence of apoptotic cells in 10 mg/kg and 20 mg/kg cisplatin treated mouse kidneys. Scale bar: 40 nm.

FIG. 31 is a set of representative images of Periodic acid—Schiff (PAS) stained renal tissues showing the intact glomerular morphologies in 10 mg/kg and 20 mg/kg cisplatin-treated mice. Scale bar: 20 μm.

FIG. 32 is a set of representative images of H&E-stained renal tissues showing the tubular necrosis (pointed by white triangles) and formation of protein casts (pointed by white arrows) in mg/kg cisplatin-treated mice. Scale bar: 50 μm.

FIG. 33 is a set of representative images of Periodic acid—Schiff (PAS) stained renal tissues showing the formation of protein casts (pointed by white arrows) in 20 mg/kg cisplatin-treated mice. Scale bar: 50 μm.

FIG. 34A is a set of bladder fluorescence images at 30 min post injection of ICG-PEG45 proved the hampered urinary clearance in 10 mg/kg cisplatin-treated mice.

FIG. 34B is a set of bladder fluorescence images at 30 min post injection of IRDye-PEG45 indicated the rapid urinary clearance in 10 mg/kg cisplatin-treated mice.

FIG. 34C is a graph showing renal clearance efficiency of ICG-PEG45 and IRDye-PEG45 in normal and 10 mg/kg cisplatin-treated mice.

FIG. 34D is a bar graph showing ex vivo kidney fluorescence intensity at 30 min p.i.

FIG. 34E is a bar graph showing blood accumulation of ICG-PEG45 and IRDye-PEG45 at min p.i.

FIG. 34F is a set of fluorescence images of kidney frozen sections at 30 min post injection of ICG-PEG45.

FIG. 34G is a graph showing the quantified intra-kidney distribution of ICG-PEG45 at 30 min p.i. Scale bar: 40 μm.

### DETAILED DESCRIPTION

Indocyanine green (ICG) is a clinically approved near-infrared (NIR)-emitting fluorophore and has been successfully applied in the fluorescence-guided surgery of breast, liver, brain cancers. However, hundreds of clinical case studies show that introducing ICG in partial nephrectomy surgery for kidney cancer has not resulted in significant improvement in reducing positive margin rates because ICG fails in hyperfluorescent imaging of kidney cancers due to its rapid hepatobiliary clearance and limited interactions with kidney cancerous tissues. In fact, ICG is just temporally (<15 min) retained in and lights up the normal renal parenchyma before it is rapidly eliminated through the liver. Additionally, the green color of ICG “bleeds” in the normal kidney tissues when excising the tumor mass, making it difficult to differentiate the tumor margins during surgery. These limitations of ICG in imaging of kidney cancer raise a question of whether ICG can be tailored to be eliminated through the renal tubules to selectively target the kidney cancers.

The present disclosure provides renal tubule-secretable ICG through PEGylation. By conjugating ICG with PEG, it was found that PEGylation enabled ICG to be rapidly and actively eliminated almost exclusively through the renal tubular secretion pathway into the urine. PEGylation prevented ICG from being taken up by the liver while enhancing its interaction with transporters of the proximal tubular cells and allowing ICG to be transported from peritubular capillary to proximal tubular lumen with assistance of organic anion transporters on the basolateral side (“enter in” proximal tubular cells) and P-glycoprotein (P-gP) efflux transporters (“get out” from the proximal tubular cells). Since P-gP efflux transporters are expressed at a much lower level on the membrane of kidney cancer cells than normal proximal tubular cells, the ICG-PEG conjugate was efficiently eliminated out of the normal kidney tissues while being retained in kidney cancerous tissues. In contrast, ICG eliminated through either the liver or glomeruli failed to selectively target kidney cancers over normal kidney tissues due to their limited interactions with transporters of proximal tubules. Not limited to primary kidney cancers, the ICG-PEG conjugate also fluorescently detected extrarenal metastases in bone, brain and lung with high specificity.

### Definitions

Unless defined otherwise, all technical and scientific terms used herein have the same meaning as is commonly understood by one of skill in the art to which the claimed subject matter belongs. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

Definition of standard chemistry terms may be found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein, which will be limited only by the appended claims.

All publications and patents mentioned herein are incorporated herein by reference in their entirety for the purpose of describing and disclosing, for example, the constructs and methodologies that are described in the publications, which might be used in connection with the methods, compositions and compounds described herein. The publications discussed herein are provided solely for their disclosure prior to the filing date of the present application. Nothing herein is to be construed as an admission that the inventors described herein are not entitled to antedate such disclosure by virtue of prior invention or for any other reason.

An “alkyl” group refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group that has at least one carbon-carbon double bond, and an “alkyne” moiety refers to a group that has at least one carbon-carbon triple bond. The alkyl moiety, whether saturated or unsaturated, may be branched, straight chain, or cyclic. Depending on the structure, an alkyl group can be a monoradical or a diradical (i.e., an alkylene group). The alkyl group could also be a “lower alkyl” having 1 to 6 carbon atoms.

As used herein, C₁-Cₓ includes, but is not limited to, C₁-C₂, C₁-C₃, C₁-C₄, C₁-C₅, C₁-C₆, C₂-C₃, C₂-C₄, C₂-C₅, C₂-C₆, C₃-C₄, C₃-C₅, C₃-C₆, C₄-C₅, C₄-C₆, and C₅-C₆.

The “alkyl” moiety may have 1 to 10 carbon atoms (whenever it appears herein, a numerical range such as “1 to 10” refers to each integer in the given range; e.g., “1 to 10 carbon atoms” means that the alkyl group may have 1 carbon atom, 2 carbon atoms, 3 carbon atoms, etc., up to and including 10 carbon atoms, although the present definition also covers the occurrence of the term “alkyl” where no numerical range is designated). The alkyl group of the compounds described herein may be designated as “C₁-C₄ alkyl” or similar designations. By way of example only, “C₁-C₄ alkyl” indicates that there are one to four carbon atoms in the alkyl chain, i.e., the alkyl chain is selected from among methyl, ethyl, propyl, iso-propyl, n-butyl, iso-butyl, sec-butyl, and t-butyl. Thus C₁-C₄ alkyl includes C₁-C₂ alkyl and C₁-C₃ alkyl. Alkyl groups can be substituted or unsubstituted. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like.

As used herein, the term “non-cyclic alkyl” refers to an alkyl that is not cyclic (i.e., a straight or branched chain containing at least one carbon atom). Non-cyclic alkyls can be fully saturated or can contain non-cyclic alkenes and/or alkynes. Non-cyclic alkyls can be optionally substituted.

The term “alkenyl” refers to a type of alkyl group in which the first two atoms of the alkyl group form a double bond that is not part of an aromatic group. That is, an alkenyl group begins with the atoms —C(R)═C(R)—R, wherein R refers to the remaining portions of the alkenyl group, which may be the same or different. The alkenyl moiety may be branched, straight chain, or cyclic (in which case, it would also be known as a “cycloalkenyl” group). Depending on the structure, an alkenyl group can be a monoradical or a diradical (i.e., an alkenylene group). Alkenyl groups can be optionally substituted. Non-limiting examples of an alkenyl group include —CH═CH₂, —C(CH₃)═CH₂, —CH═CHCH₃, —C(CH₃)═CHCH₃. Alkenylene groups include, but are not limited to, —CH═CH—, —C(CH₃)═CH—, —CH═CHCH₂—, —CH═CHCH₂CH₂— and —C (CH₃)═CHCH₂—. Alkenyl groups could have 2 to 10 carbons. The alkenyl group could also be a “lower alkenyl” having 2 to 6 carbon atoms.

The term “alkynyl” refers to a type of alkyl group in which the first two atoms of the alkyl group form a triple bond. That is, an alkynyl group begins with the atoms —C≡C—R, wherein R refers to the remaining portions of the alkynyl group, which may be the same or different. The “R” portion of the alkynyl moiety may be branched, straight chain, or cyclic. Depending on the structure, an alkynyl group can be a monoradical or a diradical (i.e., an alkynylene group). Alkynyl groups can be optionally substituted. Non-limiting examples of an alkynyl group include, but are not limited to, —C≡CH, —C≡CCH₃, —C≡CCH₂CH₃, and —C≡CCH₂—. Alkynyl groups can have 2 to 10 carbons. The alkynyl group could also be a “lower alkynyl” having 2 to 6 carbon atoms.

An “alkoxy” group refers to a (alkyl)O— group, where alkyl is as defined herein.

“Hydroxyalkyl” refers to an alkyl radical, as defined herein, substituted with at least one hydroxy group. Non-limiting examples of a hydroxyalkyl include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl.

“Alkoxyalkyl” refers to an alkyl radical, as defined herein, substituted with an alkoxy group, as defined herein.

An “alkenyloxy” group refers to a (alkenyl)O— group, where alkenyl is as defined herein.

The term “alkylamine” refers to the —N(alkyl)ₓH_{y} group, where x and y are selected from among x=1, y=1 and x=2, y=0. When x=2, the alkyl groups, taken together with the N atom to which they are attached, can optionally form a cyclic ring system.

“Alkylaminoalkyl” refers to an alkyl radical, as defined herein, substituted with an alkylamine, as defined herein.

An “amide” is a chemical moiety with the formula —C(O)NHR or —NHC(O)R, where R is selected from among alkyl, cycloalkyl, aryl, heteroaryl (bonded through a ring carbon) and heteroalicyclic (bonded through a ring carbon). An amide moiety may form a linkage between an amino acid or a peptide molecule and a compound described herein, thereby forming a prodrug. Any amine, or carboxyl side chain on the compounds described herein can be amidified. The procedures and specific groups to make such amides are known to those of skill in the art and can readily be found in reference sources such as Greene and Wuts, Protective Groups in Organic Synthesis, 3^{rd} Ed., John Wiley & Sons, New York, NY, 1999, which is incorporated herein by reference in its entirety.

The term “ester” refers to a chemical moiety with formula —COOR, where R is selected from among alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3^{rd} Ed., John Wiley & Sons, New York, NY, 1999, which is incorporated herein by reference in its entirety.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can be monocyclic or polycyclic.

As used herein, the term “ring system” refers to one, or more than one ring.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “fused” refers to structures in which two or more rings share one or more bonds.

The term “carbocyclic” or “carbocycle” refers to a ring wherein each of the atoms forming the ring is a carbon atom. Carbocycle includes aryl and cycloalkyl. The term thus distinguishes carbocycle from heterocycle (“heterocyclic”) in which the ring backbone contains at least one atom which is different from carbon (i.e., a heteroatom). Heterocycle includes heteroaryl and heterocycloalkyl. Carbocycles and heterocycles can be optionally substituted.

The term “aromatic” refers to a planar ring having a delocalized π-electron system containing 4n+2 π electrons, where n is an integer. Aromatic rings can be formed from five, six, seven, eight, nine, or more than nine atoms. Aromatics can be optionally substituted. The term “aromatic” includes both carbocyclic aryl (e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

An “aryloxy” group refers to an (aryl)O— group, where aryl is as defined herein.

“Aralkyl” means an alkyl radical, as defined herein, substituted with an aryl group. Non-limiting aralkyl groups include, benzyl, phenethyl, and the like.

“Aralkenyl” means an alkenyl radical, as defined herein, substituted with an aryl group, as defined herein.

The term “cycloalkyl” refers to a monocyclic or polycyclic radical that contains only carbon and hydrogen, and may be saturated, partially unsaturated, or fully unsaturated. Cycloalkyl groups include groups having from 3 to 10 ring atoms. Illustrative examples of cycloalkyl groups include the following moieties:

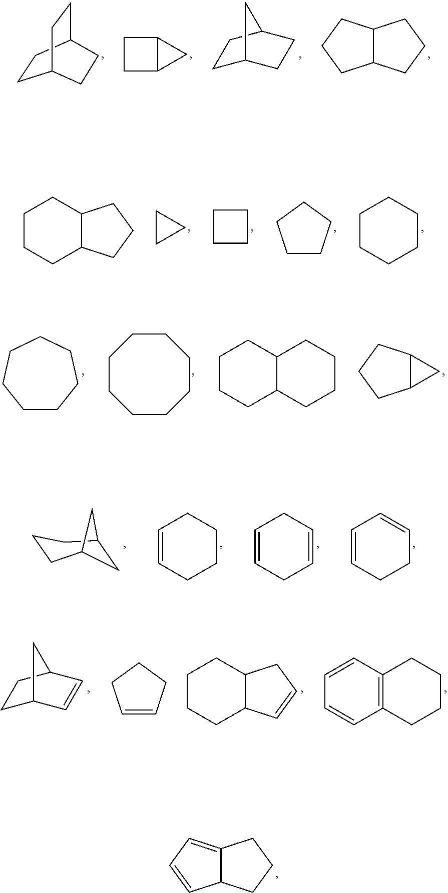

and the like. Depending on the structure, a cycloalkyl group can be a monoradical or a diradical (e.g., a cycloalkylene group). The cycloalkyl group could also be a “lower cycloalkyl” having 3 to 8 carbon atoms.

“Cycloalkylalkyl” means an alkyl radical, as defined herein, substituted with a cycloalkyl group. Non-limiting cycloalkylalkyl groups include cyclopropylmethyl, cyclobutylmethyl, cyclopentylmethyl, cyclohexylmethyl, and the like.

The term “heterocycle” refers to heteroaromatic and heteroalicyclic groups containing one to four heteroatoms each selected from O, S and N, wherein each heterocyclic group has from 4 to 10 atoms in its ring system, and with the proviso that the ring of said group does not contain two adjacent O or S atoms. Herein, whenever the number of carbon atoms in a heterocycle is indicated (e.g., C₁-C₆ heterocycle), at least one other atom (the heteroatom) must be present in the ring. Designations such as “C₁-C₆ heterocycle” refer only to the number of carbon atoms in the ring and do not refer to the total number of atoms in the ring. It is understood that the heterocylic ring can have additional heteroatoms in the ring. Designations such as “4-6 membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). In heterocycles that have two or more heteroatoms, those two or more heteroatoms can be the same or different from one another. Heterocycles can be optionally substituted. Binding to a heterocycle can be at a heteroatom or via a carbon atom. Non-aromatic heterocyclic groups include groups having only 4 atoms in their ring system, but aromatic heterocyclic groups must have at least 5 atoms in their ring system. The heterocyclic groups include benzo-fused ring systems. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one. Depending on the structure, a heterocycle group can be a monoradical or a diradical (i.e., a heterocyclene group).

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. Illustrative examples of heteroaryl groups include the following moieties:

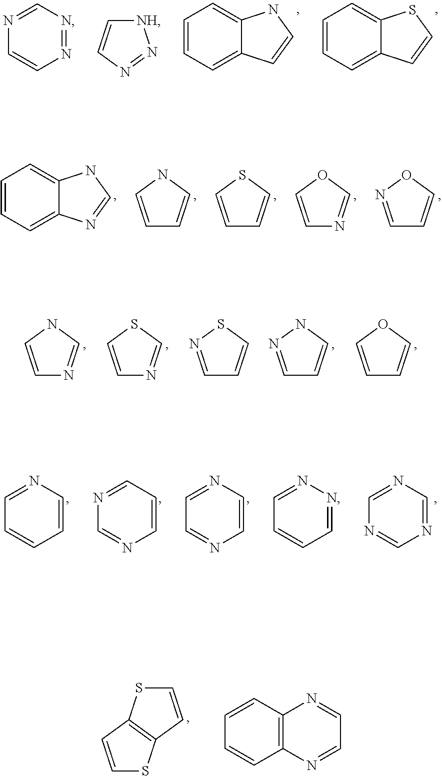

and the like. Depending on the structure, a heteroaryl group can be a monoradical or a diradical (i.e., a heteroarylene group).

As used herein, the term “non-aromatic heterocycle”, “heterocycloalkyl” or “heteroalicyclic” refers to a non-aromatic ring wherein one or more atoms forming the ring is a heteroatom. A “non-aromatic heterocycle” or “heterocycloalkyl” group refers to a cycloalkyl group that includes at least one heteroatom selected from nitrogen, oxygen and sulfur. The radicals may be fused with an aryl or heteroaryl. Heterocycloalkyl rings can be formed by three, four, five, six, seven, eight, nine, or more than nine atoms. Heterocycloalkyl rings can be optionally substituted. In certain embodiments, non-aromatic heterocycles contain one or more carbonyl or thiocarbonyl groups such as, for example, oxo- and thio-containing groups. Examples of heterocycloalkyls include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Illustrative examples of heterocycloalkyl groups, also referred to as non-aromatic heterocycles, include:

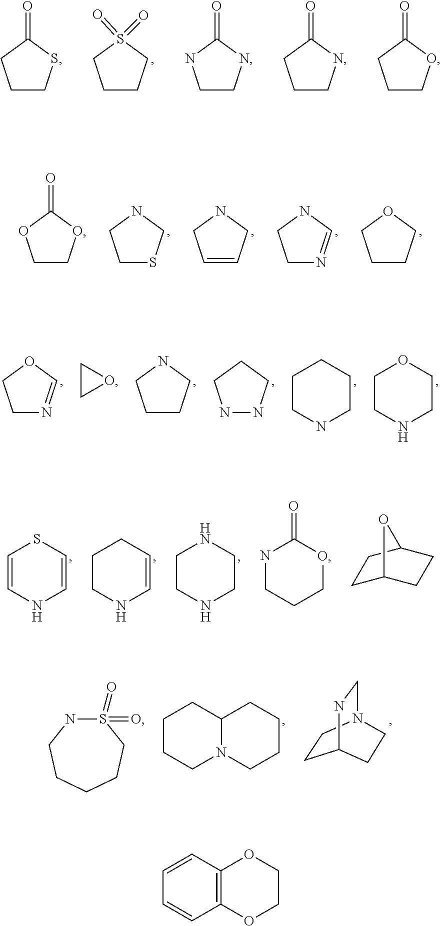

and the like. The term heteroalicyclic also includes all ring forms of the carbohydrates, including but not limited to the monosaccharides, the disaccharides and the oligosaccharides. Depending on the structure, a heterocycloalkyl group can be a monoradical or a diradical (i.e., a heterocycloalkylene group).

The term “halo” or, alternatively, “halogen” or “halide” means fluoro, chloro, bromo and iodo.

The terms “haloalkyl,” “haloalkenyl,” “haloalkynyl” and “haloalkoxy” include alkyl, alkenyl, alkynyl and alkoxy structures in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another.

The term “fluoroalkyl,” as used herein, refers to alkyl group in which at least one hydrogen is replaced with a fluorine atom. Examples of fluoroalkyl groups include, but are not limited to, —CF₃, —CH₂CF₃, —CF₂CF₃, —CH₂CH₂CF₃ and the like.

As used herein, the terms “heteroalkyl” “heteroalkenyl” and “heteroalkynyl” include optionally substituted alkyl, alkenyl and alkynyl radicals in which one or more skeletal chain atoms is a heteroatom, e.g., oxygen, nitrogen, sulfur, silicon, phosphorus or combinations thereof. The heteroatom(s) may be placed at any interior position of the heteroalkyl group or at the position at which the heteroalkyl group is attached to the remainder of the molecule. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In addition, up to two heteroatoms may be consecutive, such as, by way of example, —CH₂—NH—OCH₃ and —CH₂—O—Si(CH₃)₃.

The term “heteroatom” refers to an atom other than carbon or hydrogen. Heteroatoms are typically independently selected from among oxygen, sulfur, nitrogen, silicon and phosphorus, but are not limited to these atoms. In embodiments in which two or more heteroatoms are present, the two or more heteroatoms can all be the same as one another, or some or all of the two or more heteroatoms can each be different from the others.

The term “bond” or “single bond” refers to a chemical bond between two atoms, or two moieties when the atoms joined by the bond are considered to be part of larger substructure.

The term “moiety” refers to a specific segment or functional group of a molecule. Chemical moieties are often recognized chemical entities embedded in or appended to a molecule.

A “thioalkoxy” or “alkylthio” group refers to a —S-alkyl group.

A “alkylthioalkyl” group refers to an alkyl group substituted with a —S-alkyl group.

As used herein, the term “O-carboxy” or “acyloxy” refers to a group of formula RC(═O)O—.

“Carboxy” means a —C(O)OH radical.

As used herein, the term “acetyl” refers to a group of formula —C(═O)CH₃.

“Acyl” refers to the group —C(O)R.

As used herein, the term “cyano” refers to a group of formula —CN.

As used herein, the substituent “R” appearing by itself and without a number designation refers to a substituent selected from among from alkyl, cycloalkyl, aryl, heteroaryl (bonded through a ring carbon) and non-aromatic heterocycle (bonded through a ring carbon).

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, alkylthio, arylthio, alkylsulfoxide, arylsulfoxide, alkylsulfone, arylsulfone, cyano, halo, acyl, nitro, haloalkyl, fluoroalkyl, amino, including mono- and di-substituted amino groups, and the protected derivatives thereof. By way of example an optional substituents may be Las, wherein each Ls is independently selected from a bond, —O—, —C(=O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, -(substituted or unsubstituted C₁-C₆ alkyl), or -(substituted or unsubstituted C₂-C₆ alkenyl); and each Rₛ is independently selected from H, (substituted or unsubstituted C₁-C₄alkyl), (substituted or unsubstituted C₃-C₆cycloalkyl), heteroaryl, or heteroalkyl. The protecting groups that may form the protective derivatives of the above substituents are known to those of skill in the art and may be found in references such as Greene and Wuts, above.

The term “acceptable” or “pharmaceutically acceptable”, with respect to a formulation, composition or ingredient, as used herein, means having no persistent detrimental effect on the general health of the subject being treated or does not abrogate the biological activity or properties of the compound, and is relatively nontoxic.

The term “subject” as used herein, refer to either a human or a non-human animal. The term “subject” thus includes mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as pain, e.g., neuropathic pain. The skilled worker can readily determine the effective amount for a given situation by routine experimentation.

As used herein, the term “about” means a range of values that are similar to the stated reference value. In certain embodiments, the term “about” refers to a range of values that fall within 10 percent or less (e.g., 10%, 9%, 8%, 7%, 6%, 5%, 4%, 3%, 2%, or 1%) of the stated reference value.

As used herein, the term “significantly” means at least 5%, at least 10%, at least 15%, at least 20%, at least 25%, at least 30%, at least 35%, at least 40%, at least 45%, or at least 50%.

As used herein, the term “ICG-PEG conjugate” refers to a composition comprising PEG and ICG, wherein PEG is conjugated to ICG. In some embodiments, the composition comprises a secondary moiety that is conjugated to PEG or ICG.

As used herein, the term “biochemically activatable agent” refers to an agent that can selectively react with biomolecules, enzymes, or ions.

### ICG-PEG Conjugates

In one aspect, the present disclosure provides ICG-PEG conjugates.

In certain embodiments, the ICG-PEG conjugate is of formula I:

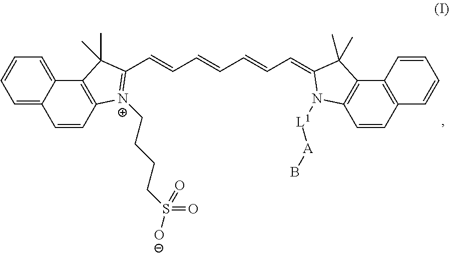

or a pharmaceutically acceptable salt thereof, wherein:
L¹ is independently optionally substituted alkylene, haloalkylene, alkenylene or alkynylene;
A is independently —C(O)NH(CH₂CH₂O)ₙ—, —C(O)O(CH₂CH₂O)ₙ—, —C(O)S(CH₂CH₂O)ₙ—, —NHC(O)CH₂O(CH₂CH₂O)ₙ—, —OC(O)CH₂O(CH₂CH₂O)ₙ—, or —SC(O)CH₂O(CH₂CH₂O)ₙ—, wherein the (CH₂CH₂O)— end is connected to B;
n is an integer selected from about 10 to about 1000; and
B is independently H, or optionally substituted alkyl.

In certain embodiments, L¹ is unsubstituted C₁₋₆ alkylene or C₁₋₆ haloalkylene.

In certain embodiments, B is H or unsubstituted C₁₋₆ alkyl.

In certain embodiments, B is C₁₋₆ alkyl substituted with one or more —OH, —NH₂, —SH, or —COOH.

In certain embodiments, B is —CH₂CH₂OH, —CH₂CH₂NH₂, —CH₂CH₂SH, —CH₂CH₂C(O)OH, or —CH₂C(O)OH.

In certain embodiments, the ICG-PEG conjugate is of the formula

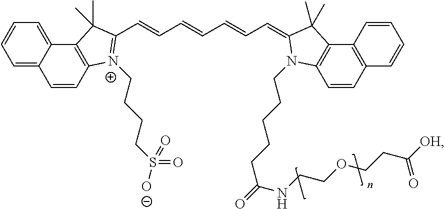

or a pharmaceutically acceptable salt thereof.

In certain embodiments, n is at least about 10, at least about 14, at least about 18, at least about 22, at least about 26, at least about 30, at least about 34, at least about 38, or at least about 42. In certain embodiments, n is no more than about 1000, no more than about 950, no more than about 900, no more than about 850, no more than about 800, no more than about 750, no more than about 700, no more than about 650, no more than about 600, no more than about 550, no more than about 500, no more than about 450, or no more than about 400.

Combinations of the above-referenced ranges for n are also possible (e.g., at least about to no more than 900, at least about 22 to no more than about 700), inclusive of all values and ranges therebetween.

In certain embodiments, n is an integer selected from about 22 to about 220. In certain embodiments, n is an integer selected from about 22 to about 44. In certain embodiments, n is an integer selected from about 43 to about 107, e.g., from about 43 to about 90, from about 43 to about 85, from about 43 to about 80, from about 43 to about 75, from about 43 to about 70, from about 43 to about 65, from about 43 to about 60, or from about 43 to about 55.

In certain embodiments, n is 42, 43, 44, 45, 46, 47, 48, 49, or 50. In certain embodiments, n is 22. In certain embodiments, n is 220.

In certain embodiments, PEG has a molecular weight of about 2000 Da to about 5000 Da, e.g., about 2000 Da to about 4500 Da, about 2000 Da to about 4000 Da, about 2000 Da to about 3500 Da, or about 2000 Da to about 3000 Da.

In certain embodiments, the ICG-PEG conjugate is in the form of nanoparticles. In certain embodiments, the nanoparticles have an average diameter of about 0.5 nm to about 12 nm, e.g., about 0.5 nm to about 10 nm, about 0.5 nm to about 8 nm, about 0.5 nm to about 6 nm, about 1 nm to about 12 nm, about 1 nm to about 10 nm, about 1 nm to about 8 nm, or about 1 nm to about 6 nm.

In certain embodiments, the ICG-PEG conjugate further comprises a secondary moiety conjugated to PEG or ICG. In certain embodiments, the secondary moiety is an imaging agent, biochemically activatable agent, or a therapeutic agent.

In another aspect, the present disclosure provides a pharmaceutical composition comprising an ICG-PEG conjugate and a pharmaceutically acceptable carrier.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Formulations suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

### Methods of Using ICG-PEG Conjugates

The ICG-PEG conjugates described herein can be used in a variety of applications, including, but not limited to, diagnostic and therapeutic applications.

In one aspect, the ICG-PEG conjugate described herein can be used to measure an expression level of an influx or efflux transporter in a subject. In some embodiments, the ICG-PEG conjugate can be used to identify the differences in expression of influx or efflux transporters among healthy people but with differences in gender, age, etc. This can provide useful information for personalized medicine.

Accordingly, the present disclosure provides a method of measuring an expression level of an influx or efflux transporter in a subject, comprising: (a) administering to the subject an ICG-PEG conjugate; (b) determining a concentration of the ICG-PEG conjugate in a biological sample obtained from the subject; and (c) determining the expression level of the influx or efflux transporter based on the concentration of the ICG-PEG conjugate.

The present disclosure also provides a method of measuring an expression level of an influx or efflux transporter in a subject, comprising: (a) administering to the subject an ICG-PEG conjugate; (b) measuring an intensity of a signal from the ICG-PEG conjugate in a tissue of the subject; and (c) determining the expression level of the influx or efflux transporter based on the intensity.

In certain embodiments, the expression level is the absolute expression level. In certain embodiments, the expression level is the relative expression level. For example, the expression level can be relative to that in a population with different gender and/or age.

In one aspect, the ICG-PEG conjugate described herein can be used as a marker for diagnostic applications, including but not limited to, monitoring influx transporter activities, monitoring efflux transporter activities, monitoring kidney secretion function, monitoring liver function, and diagnosing or detecting a disease or condition associated with abnormal expression of an influx or efflux transporter. In certain embodiments, the ICG-PEG conjugate described herein can be used as an exogenous marker, e.g., for blood or urine samples.

Accordingly, the present disclosure provides a method of diagnosing a disease or condition associated with abnormal expression of an influx or efflux transporter in a subject, comprising: (a) administering to the subject an ICG-PEG conjugate; (b) determining a concentration of the ICG-PEG conjugate in a biological sample obtained from the subject; (c) comparing the concentration of the ICG-PEG conjugate with a first reference level; and (d) determining that the subject has the disease or condition if the concentration of the ICG-PEG conjugate is significantly greater or lower than the first reference level.

Abnormal expression of an influx transporter can mean either upregulation or downregulation of the influx transporter as compared to the expression level in a normal tissue. Abnormal expression of an efflux transporter can mean either upregulation or downregulation of the efflux transporter as compared to the expression level in a normal tissue.

In certain embodiments, the first reference level is the concentration of the ICG-PEG conjugate in a corresponding biological sample obtained from a healthy subject. In certain embodiments, the healthy subject is of the same gender and/or similar age as the subject.

The present disclosure also provides a method of monitoring kidney secretion function of a subject, comprising: (a) administering to the subject an ICG-PEG conjugate; (b) determining a first concentration of the ICG-PEG conjugate in a first biological sample obtained from the subject at a first time point; (c) determining a second concentration of the ICG-PEG conjugate in a second biological sample obtained from the subject at a second time point, wherein the second time point is after the first time point; (d) determining renal clearance kinetics based on the first concentration and the second concentration; and (e) optionally comparing the renal clearance kinetics with a second reference level.

In certain embodiments, the first time point can be at least about 1 minute, at least about minutes, at least about one hour, at least about 90 minutes, or at least about two hours after the ICG-PEG conjugate is administered. In certain embodiments, the second time point can be at least about 5 minutes, at least about 30 minutes, at least about one hour, at least about 90 minutes, at least about two hours, or more after the first time point. To determine the renal clearance kinetics, more than two time points (e.g., three, four, or five time points) can be utilized.

In certain embodiments, the method further comprises determining that the subject has abnormal kidney secretion function if the renal clearance kinetics is significantly greater or less than the second reference level.

In certain embodiments, the second reference level is the renal clearance kinetics of a healthy subject. In certain embodiments, the healthy subject is of the same gender and/or similar age as the subject.

In certain embodiments, the biological sample can be blood, plasma, serum, urine, a tissue biopsy, a cell, a plurality of cells, fecal matter, or saliva. In certain embodiments, the biological sample is a blood sample. In certain embodiments, the biological sample is a urine sample.

Without wishing to be bound by theory, as the ICG-PEG conjugate with PEG molecular weight smaller than 2 kDa can be eliminated through both the liver and kidneys, liver injury will slow down its elimination through the liver pathway, but increase its renal clearance. Accordingly, certain ICG-PEG conjugates can be used to detect liver diseases. The present disclosure provides a method of detecting a liver disease in a subject, comprising: (a) administering to the subject an ICG-PEG conjugate, wherein PEG has a molecular weight of less than 2 kDa; (b) determining a concentration of the ICG-PEG conjugate in a urine sample obtained from the subject; (c) comparing the concentration of the ICG-PEG conjugate with a third reference level; and (d) determining that the subject has the liver disease when the concentration of the ICG-PEG conjugate is significantly less than the third reference level. In certain embodiment, the ICG-PEG conjugate with PEG molecular weight between about 100 Da and about 2 kDa can be eliminated through both the liver and kidneys. Accordingly, in certain embodiments, the PEG in the conjugate used for detecting liver diseases has a molecular weight of at least about 100 Da to less than about 2 kDa, e.g., between about 100 Da and about 1800 Da, between about 100 Da and about 1600 Da, between about 100 Da and about 1400 Da, between about 100 Da and about 1200 Da, between about 100 Da and about 1000 Da, between about 100 Da and about 800 Da, between about 100 Da and about 600 Da, between about 200 Da and about 1800 Da, between about 200 Da and about 1600 Da, between about 200 Da and about 1400 Da, between about 200 Da and about 1200 Da, between about 200 Da and about 1000 Da, between about 200 Da and about 800 Da, or between about 200 Da and about 600 Da.

In certain embodiments, the third reference level is the concentration of the ICG-PEG conjugate in a urine sample obtained from a healthy subject. In certain embodiments, the healthy subject is of the same gender and/or similar age as the subject.

In another aspect, the ICG-PEG conjugate described herein can be used as an imaging agent configured to produce a signal with measurable intensity. By measuring the intensity of a signal from the ICG-PEG conjugate after it is administered to a subject, it permits a technician/physician to identify diseased tissues, monitor influx transporter activities, monitor efflux transporter activities, or detect a disease or condition associated with abnormal expression of an influx or efflux transporter.

In certain embodiments, measuring an intensity of a signal from the ICG-PEG conjugate in a tissue merely measures the intensity without spatial information.

In certain embodiments, measuring an intensity of a signal from the ICG-PEG conjugate in a tissue comprises imaging the tissue so as to produce both the intensity and spatial information of the ICG-PEG in the tissue. In certain embodiments, the ICG-PEG conjugate can be used as a positive image contrast agent.

Accordingly, the present disclosure provides a method of monitoring kidney secretion function of a subject, comprising: (a) administering to the subject an ICG-PEG conjugate; (b) measuring, at a first time point, a first intensity of a signal from the ICG-PEG conjugate in a tissue of the subject; and (c) measuring, at a second time point, a second intensity of a signal from the ICG-PEG conjugate in the tissue of the subject. This method can thus permit temporal monitoring of kidney secretion function.

In certain embodiments, the method of monitoring kidney secretion function further comprises comparing the second intensity with the first intensity and determining that the kidney secretion function is abnormal or deteriorating if the second intensity is significantly higher or lower than the first intensity.

In certain embodiments, the method of monitoring kidney secretion function further comprises determining renal clearance kinetics based on the first intensity and the second intensity; and (e) optionally comparing the renal clearance kinetics with the second reference level, as discussed above.

In certain embodiments, the first time point can be at least about 1 minute, at least about minutes, at least about one hour, at least about 90 minutes, or at least about two hours after the ICG-PEG conjugate is administered. In certain embodiments, the second time point can be at least about 5 minutes, at least about 30 minutes, at least about one hour, at least about 90 minutes, at least about two hours, or more after the first time point. The method of monitoring kidney secretion function can utilize more than two time points (e.g., three, four, or five time points) to determine the intensity kinetics. Each time point can be separated from its prior time point by at least about 5 minutes, at least about 30 minutes, at least about one hour, at least about minutes, at least about two hours, or more.

In certain embodiments, the intensity is measured as frequently as needed, e.g., every three hours, every 2.5 hours, every two hours, every 1.5 hours, every hour, every 30 minutes, every 20 minutes, every 10 minutes, or every 5 minutes.

In certain embodiments, the measurements are performed for as long as needed, e.g., in a few hours to a few weeks, e.g., about 6 hours, about 12 hours, about 18 hours, about 24 hours, about two days, about four days, about eight days, or about 16 days.

In certain embodiments, the method of monitoring kidney secretion function further comprises determining that the subject has abnormal kidney secretion function if the renal clearance kinetics is significantly greater or less than the second reference level. Abnormal kidney secretion function can then be used to diagnose a disease or condition associated with abnormal expression of an influx or efflux transporter.

The present disclosure also provides a method of diagnosing a disease or condition associated with abnormal expression of an influx or efflux transporter in a subject, comprising: (a) administering to the subject an ICG-PEG conjugate; (b) measuring an intensity of a signal from the ICG-PEG conjugate in a tissue of the subject; (c) comparing the intensity with a fourth reference level; and (d) determining that the subject has the disease or condition if the intensity is significantly greater or lower than the fourth reference level.

In certain embodiments, measuring the intensity comprises imaging the tissue , which provides a contrast index of at least about 1.5. In certain embodiments, the fourth reference level is the intensity of a signal from ICG-PEG in a corresponding normal tissue of the subject or a healthy subject. In certain embodiments, the healthy subject is of the same gender and/or similar age as the subject.

In certain embodiments, the intensity is significantly greater or lower than the fourth reference level due to either upregulated or downregulated transporters compared to nearby normal tissues or normal status.

Measuring the intensity can utilize fluorescence imaging, photoacoustic imaging, computed tomography (CT), positron emission tomography (PET), single-photon emission computerized tomography (SPECT), or magnetic resonance imaging (MRI). The tissue can be blood, a body part or a portion thereof, an organ or a portion thereof, or a diseased tissue such as a tumor. For example, the organ can be kidney or bladder. Measuring the intensity can be performed either invasively (i.e., on a biological sample obtained from a subject) or noninvasively. With respect to noninvasive measurements, a variety of devices can be used to measure the intensity of the signal from the ICG-PEG conjugate. For example, a transdermal optical device (e.g., a finger clip oximeter) can be used. In certain embodiments, a portable transdermal optical device is used to measure the intensity of the signal from the ICG-PEG conjugate.

Depending on the method used to measure the signal, the signal can be an optical signal (e.g., fluorescence), an ultrasonic signal, a radioactive signal (e.g., X-ray signal or gamma-ray signal), or a radio wave.

Measuring the intensity can provide temporal and/or spatial information of the tissue.

In yet another aspect, the present disclosure provides a method of treating a disease or condition associated with abnormal expression of an influx or efflux transporter in a subject in need thereof, comprising administering to the subject an ICG-PEG conjugate.

Depending on the composition of the ICG-PEG conjugate, the treatment methods can vary. For example, ICG is known to be a photothermal or photodynamic agent, so after the ICG-PEG conjugate is administered, electromagnetic radiation can be applied to the subject to treat the disease or condition through photothermal or photodynamic therapy.

In certain embodiments of any one of the above aspects, influx transporters include, but are not limited to, organic anion transporter family (OATs, such as OAT1, OAT2, OAT3 and OAT4), organic anion transporting polypeptides (OATPs, such as OATP4A1 and OATP4C1), organic cation transporters family (OCTs, such as OCT2, OCT3), equilibrative nucleoside transporter 1 and 2 (ENT1 and ENT2), and organic solute transporter α and β (OSTα and OSTβ).

In certain embodiments of any one of the above aspects, efflux transporters include, but are not limited to, P-glycoprotein (P-gP; also termed multidrug resistance protein 1 (MDR1)), multidrug-resistant protein 2 and 4 (MRP2 and MRP4), organic cation transporters (OCTs, such as novel OCT (OCTN)1, OCTN2, multidrug and toxin exclusion (MATE) 1, and MATE kidney-specific 2), organic anion-transporting polypeptide family (OATPs), breast cancer resistance protein (BCRP), and organic anion transporter 4 (OAT4).

Other influx/efflux transporters: transporters of peptides, PDZ Domains, Type 1 Sodium/Phosphate Co-Transport (NPT1), URAT1, BSP/Bilirubin binding protein (BBBP).

In certain embodiments of any one of the above aspects, the subject has upregulated or downregulated expression of P-glycoprotein (P-gp), multidrug-resistant protein 2 (MRP2), MRP4, an organic cation transporter (OCT), an organic anion transporter (OAT), an organic anion-transporting polypeptide (OATP), breast cancer resistance protein (BCRP), or organic anion transporter 4 (OAT4), equilibrative nucleoside transporter 1 (ENT1), ENT2, organic solute transporter α (OSTα), or OSTβ.

In certain embodiments of any one of the above aspects, the disease or condition associated with abnormal expression of an influx or efflux transporter is renal tubular secretion dysfunction or renal tubular injury.

In certain embodiments of any one of the above aspects, the renal tubular secretion dysfunction or renal tubular injury is proximal renal tubular secretion dysfunction or proximal renal tubular injury.

In certain embodiments of any one of the above aspects, the renal tubular secretion dysfunction or renal tubular injury is associated with a kidney disease or condition selected from acute kidney injury, chronic kidney injury, kidney cancer, lupus nephritis, diabetes-induced kidney injury, polycystic kidney disease, sepsis, kidney inflammation, kidney transplant rejection, and kidney dysfunction or kidney injury caused by diseases in other tissues and organs such as cancer and liver diseases.

In certain embodiments of any one of the above aspects, the disease or condition associated with abnormal expression of an influx or efflux transporter is kidney cancer, breast cancer, liver cancer, ovarian cancer, bladder cancer, prostate cancer, lung cancer, pancreatic cancer, bone cancer, or colon cancer, or their metastases in other organs or normal tissues.

In certain embodiments, the kidney cancer is renal cell carcinoma or renal oncocytoma, or their metastases in other organs or normal tissues.

In certain embodiments, the kidney cancer is renal cell carcinoma. In certain embodiments, the renal cell carcinoma is clear cell renal cell carcinoma (ccRCC), or papillary RCC (pRCC).

In certain embodiments, the breast cancer is triple negative breast cancer, or their metastases in other organs or normal tissues. In certain embodiments, the triple negative breast cancer is 4T1 or MCF-7 triple negative breast.

In certain embodiments of any one of the above aspects, the ICG-PEG conjugate is administered intravenously, intraperitoneally, subcutaneously, or intraarterially. In certain embodiments of any one of the above aspects, the ICG-PEG conjugate is administered intravenously.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Experimental Procedures

### Materials and Equipment

PEG samples with average molecular weight of 1100 Da, 2100 Da, 3500 Da, 5000 Da and 10100 Da were purchased from Sigma-Aldrich (USA). ICG-NHS and IRDye800CW-NHS were purchased from Intrace Medical (Switzerland) and LI-COR, respectively. Absorption spectra were measured by a Virian 50 Bio UV-vis spectrophotometer. Fluorescence spectra were acquired by a PTI QuantaMaster™ 30 Fluorescence Spectrophotomer (Birmingham, NJ). In vivo fluorescence images were recorded using a Carestream In-vivo FX Pro imaging system. Optical images of cultured cells and tissue slides were obtained with an Olympus IX-71 inverted fluorescence microscope coupled with Photon Max 512 CCD camera (Princeton Instruments). Agarose gel electrophoresis was carried out by a Bio-Rad Mini-Sub Cell GT system. Animal studies were performed according to the guidelines of the University of Texas System Institutional Animal Care and Use Committee. BALB/c mice (BALB/cAnNCr, strain code 047) of 6-8 weeks old, weighing 20-25g, were purchased from Envigo. Nude mice (Athymic NCr-nu/nu, strain code 069) of 6-8 weeks old, weighing 20-25 g, were also purchased from Envigo. All of these mice were randomly allocated and housed under standard environmental conditions (23±1° C., 50±5% humidity and a 12/12 h light/dark cycle) with free access to water and standard laboratory food.

### Synthesis of ICG-PEG Conjugates

400 μL, 10 mM PEG molecule in ultrapure water was added into 400 μL, 400 μM ICG-NHS in DMSO and the mixture was vortexed for 3 h. Then ICG-PEG conjugates were purified with sephadex column from unconjugated ICG and PEG molecule with mobile phase of ultrapure water. The different mobilities of ICG, ICG-PEG22, ICG-PEG45 and ICG-PEG220 in sephadex column and agarose gel both proved the successful synthesis of ICG-PEG conjugates. The IRDye800CW-PEG45 is synthesized in a similar way with ICG-PEG45 and detailed procedures were reported previously.

### Serum Protein Binding Test

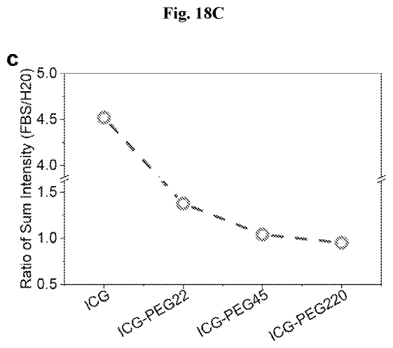

To test whether the PEG conjugation will affect the protein binding of ICG or not, free ICG, ICG-PEG22, ICG-PEG45 and ICG-PEG220 were incubated with either phosphate-buffered saline (PBS) or PBS supplemented with 10% (v/v) fetal bovine serum (FBS) at 37° C. for 30 min. In order to identify the colorless protein band, FBS incubated ICG and ICG-PEGn were stained by 10% (v/v) Coomassie Brilliant Blue 250 (CBB 250). All these samples were analyzed using 2% agarose gel electrophoresis. In addition to gel electrophoresis, the protein binding of ICG and ICG-PEGn was also tested by quantifying their fluorescence intensity in water and in 100% FBS (same concentration, but different solvent), as shown in FIGS. 18A-18C. The fluorescence images were taken by Carestream In-vivo FX Pro imaging system under 790 nm/830 nm. Non-invasive fluorescence imaging of body with ICG-PEG conjugates and ex vivo images

Hair-removed BALB/c mouse (˜25 g/mouse) was prone-positioned on imaging stage of Carestream In-vivo FX Pro imaging system under 3% isoflurane anesthesia and then intravenously injected by 200 μL, 40 μM ICG or ICG-PEG conjugates, following a start of sequential time-series imaging collection for 10 min. The fluorescence imaging parameters were set as follow: EX760/EM830 nm; 10 sec exposure time; 2×2 binning. At 10 min post injection, liver, kidneys, heart, spleen and urine in bladder were harvested and were imaged under EX790/EM830 nm, 10 sec exposure.

### Clearance Efficiencies of ICG-PEG Conjugates in Urine and Feces

BALB/c mice were intravenously injected with ICG (n=3), ICG-PEG conjugates (n=3 for each conjugate), respectively, with concentration of 40 μM and injection volume of 200 μL and then placed in metabolism cages. The separated mouse urine and feces from ICG, ICG-PEG22, ICG-PEG45 and ICG-PEG220 were collected at 24 h post injection. Then, the urine and feces were quantified based on fluorescence and each standard curve of conjugates were built in control urine and control feces.

### Noninvasive Fluorescence Imaging of Kidney With ICG-PEG45

The hair-removed BALB/c mouse was anesthetized using 3% isoflurane and a catheter filled with PBS was inserted into the tail vein. The mouse with tail vein catheter was placed in supine position on the imaging stage of Carestream In-vivo FX Pro imaging system, allowing the back to face the excitation light and CCD camera. Mouse with the steady breath rate of 10-14 times per 15 sec was injected PBS solution of ICG-PEG45 (200 μL, 40 μM) and then followed sequential time-series imaging (10 sec exposure) collection with EX790/EM830 nm for ICG-PEG45.

### Kidney Slides Imaging With Optical Microcopy

BALB/c mice were sacrificed at 5 min, 10 min and 1 h after intravenous administration of 200 μL, 400 μM of ICG-PEG45. And other BALB/c mice were sacrificed at 5 min and 1 h after intravenous administration of 200 μL, 400 μM of 800CW-PEG45. The kidneys were then collected and fixed immediately in 10% neutral buffered formalin, followed by standard dehydration and paraffin embedding. The embedded tissues were then sectioned into 4 μm slices and H&E stained. The final slides were visualized under Olympus IX-71 fluorescence microscope. The filters used for ICG-PEG45 are EX775/EM845 and dichroic mirror 810 nm. The filters use for 800CWPEG45 are EX747/EM780LP and dichroic mirror 776 nm.

### Probenecid Inhibition Studies

BALB/c mice were pre-treated by intraperitoneal injection of 200 mg/kg probenecid and control group were treated by PBS. After 30 min, ICG-PEG45, 800CW-PEG45 and FITC-inulin were intravenously injected into control group of mice and probenecid-treated mice, respectively, n=3 for each group. The mice were anesthetized under 3% isoflurane for 30 min under steady breath rate of 7-8 times per 15 sec. After 30 min, the urine was collected from bladder and quantified by fluorescence.

### RCC and RCC Metastasis Model Set-Up

Orthotopic model (i.e., sponge model): orthotopic model was established by performing unilateral renal implantation[2]. Briefly, the procedures were performed by a 1.0-cm dorsal incision using 6-8 weeks old NOD/SCID mouse. The cell suspension (1-2×10⁶ cells) mixed with Gelfoam® (Ethicon, Somerville, NJ) was implanted into subcapsular space of the left kidney with a capillary tube. Tumor growth was monitored by Bioluminescent imaging using IVIS® Spectrum (PerkinElmer, Waltham, MA). PDX (patients derived xenograft) model was used frozen RCC-PDX tissue samples (20-30 mm3) from UT Southwestern Kidney Cancer and SPORE program. Tissues were surgically implanted into left kidney of 6-8 weeks-old male NOD/SCID mice as previously described. Metastatic tumor models were established by intravenously injection of RCC cells through tail vein into 6-8 weeks-old male NOD/SCID mice. Tumors were monitored by Bioluminescent imaging. All experimental procedures were approved by the Institutional Animal Care and Use Committee. When above tumors were ready, 40 μM, 200 μL ICG-PEG45 were intravenously injected into mice. Normal right kidney and left kidney with RCC were collected and imaged at 24 h (orthotopic model), 68 h (PDX) after injection of ICG-PEG45. Metastatic tumors were collected and imaged at 24 h after injection of ICG-PEG45.

### Noninvasive Fluorescence Imaging of RCC-Implanted Kidney With ICG-PEG45

The hair-removed RCC-implanted NOD/SCID mouse was anesthetized using 3% isoflurane and a catheter filled with PBS was inserted into the tail vein. The mouse with tail vein catheter was placed in supine position on the imaging stage of Carestream In-vivo FX Pro imaging system, allowing the back to face the excitation light and CCD camera. The mouse was imaged at 30 min, 1 h, 3 h, 5 h, 12 h and 24 h after injection of ICG-PEG45 (200 μL, 40 μM) under EX790/EM830 nm (10 sec exposure).

### Ex Vivo RCC Fluorescent Imaging

Orthotopic papillary RCC-implanted NOD/SCID mouse was sacrificed at 24 h (PDX ccRCC, 68 h) after intravenous injection of ICGPEG45. The RCC-implanted left kidney and normal right kidney were both collected and cut into half for ex vivo fluorescent imaging under EX790/EM830 nm. Then the left RCC-implanted kidney was fixed immediately in 10% neutral buffered formalin, followed by standard dehydration and paraffin embedding. The embedded tissues were then sectioned into 4 μm slices and H&E stained. The final slides were visualized under Olympus IX-71 fluorescence microscope. The filters used for ICG-PEG45 are EX775/EM845 and dichroic mirror 810 nm.

### P-gP Expression Measurement by Western Blotting

The papillary renal cell carcinoma cell (529B), normal kidney proximal tubular cell (HK2), normal kidney tissue and RCC tissue (ACHN tumor) were lysed using radioimmunoprecipitation assay buffer (RIPA buffer, 150 mM NaCl, 1% Triton X-100, 50 mM Tris pH 8.0, 0.5% sodium deoxycholate and 0.1% sodium dodecyl sulfate containing 1% protease inhibitor cocktail [Roche, Indianapolis, IN]) and the same amount (30 μg) of protein from each sample was electrophoresized on 4-12% gradient Bolt gels (Life Technologies) then electroblotted onto nitrocellulose membranes. The membrane was incubated with 5% nonfat dry milk (w/v) for 1 h and then washed in PBS containing 0.1% Tween-20. Membranes were then incubated with designed primary monoclonal antibody (MA1-26528, Invitrogen, Carlsbad, CA) and the corresponding secondary antibodies conjugated with horseradish peroxidase (HRP) at room temperature for 1 h. The target proteins (P-gP) were detected with a fluorChem digital imaging system (Alpha Innotech, San Leandro, CA) using Western Bright Quantum HPR Substrate Kit (Advansta, Menlo Park, CA). Actin was used as an internal loading control for the measurement of P-gP expression at the cellular level and GAPDH was used as an internal loading control for the measurement of P-gP expression at the tissue level. Cellular uptake of ICG-PEG45 by HK2 and 529B before and after inhibitor treatment

The papillary renal cell carcinoma cell (529B), normal kidney proximal tubular cell (HK2) (1×105) were seeded in peri-dish (n=3) and allowed to adhere and grow for 24 h. These cells were incubated with 25 μM ICG-PEG45 with or without 20 μM cyclosporine A (Adipogen Life Sciences (San Diego, CA)) or 10 μM Tariquidar (MedChemexpress (Monmouth Junction, NJ)) for 1 hour in cell incubator. The same volume of DMSO was added as control. After 1 h incubation, the cells were washed 3 times with ice cold HEPES buffer solution and maintained with 1 mL of HEPES buffer solution for imaging. The cells were further imaged using fluorescent microscope with the ICG channel of excitation:710 nm, emission:830 nm. The exposure time were set as follow: 0.01 s for bright field; 5 s for fluorescent imaging. 4 images from 4 random areas were taken for each petri-dish. The average intensity of ICG inside each cell was quantified using ImageJ. The experiment was repeated 3 times with similar results.

### Statistical Analysis

Error bars are reported as mean±s.d. The differences between groups were compared by analysis of Student's t-test. P-value<0.05 was considered to be statistically significant. N.S. means no significant difference with p value>0.05. Investigators conducting the experiments were not blinded.

### Example 1. PEG45 in Altering the Clearance of ICG

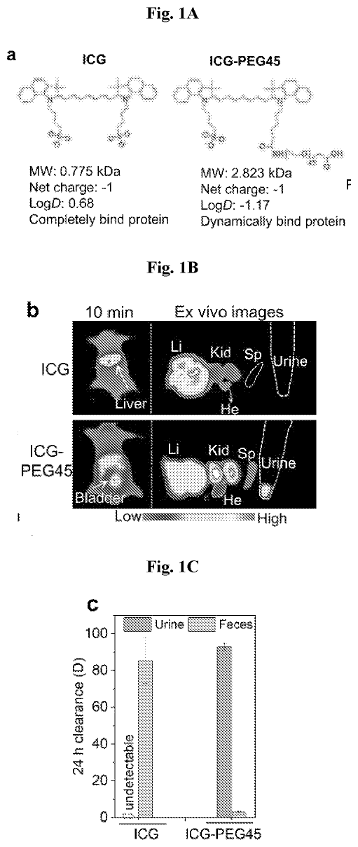

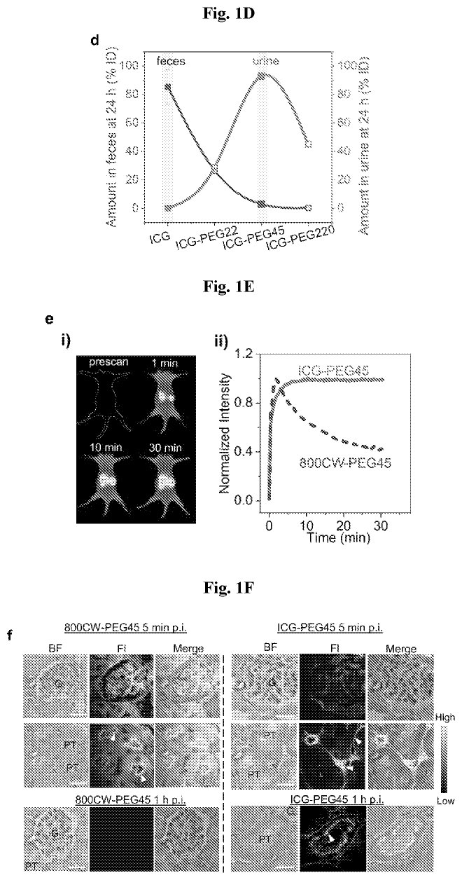

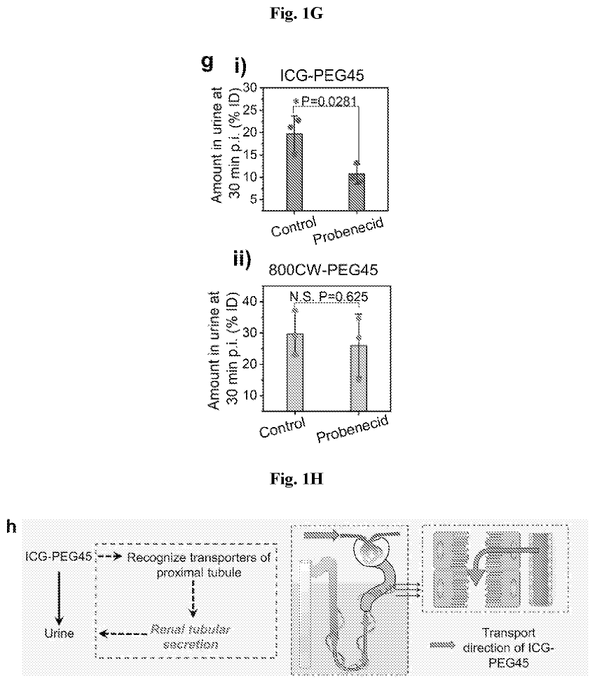

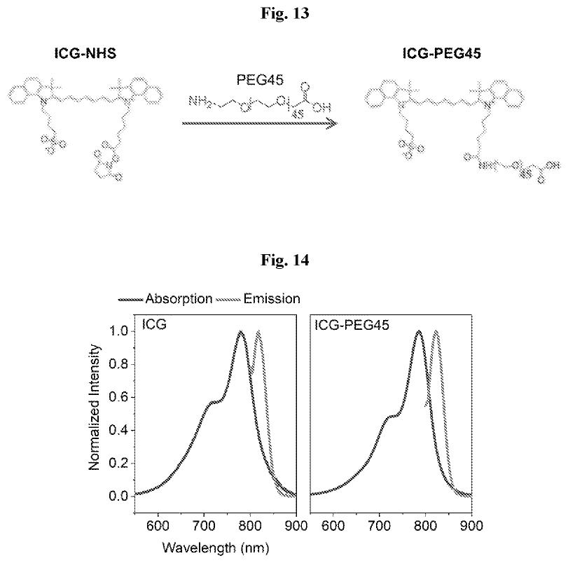

The ICG-PEG45 was readily synthesized through the reaction between the N-hydroxysuccinimide (NHS) ester of ICG and the amine group of PEG45 (MW, 2100 Da) molecules (FIG. 13). Unreacted ICG and PEG45 were removed through Sephadex-based gel filtration based on their differences in hydrophobicity and size. ICG-PEG45 exhibited the same absorption and photoluminescence properties as free ICG (FIG. 14), which allowed in situ fluorescent monitoring of its transport and tumor targeting in the kidneys. While ICG is known to strongly bind proteins, the conjugation of PEG45 molecule significantly reduced the affinity of ICG to serum proteins (FIG. 15) because of the dramatically reduced hydrophobicity of ICG-PEG45 (logD=−1.17) compared to that of free ICG (logD=0.68, FIG. 1A). Due to the strong serum protein binding, free ICG was rapidly transported into the liver upon intravenous injection, leading to little accumulation in the kidneys and undetectable signal in urine (FIG. 1B). In contrast, the conjugation of PEG45 switched the elimination organs of ICG from liver to kidney (FIG. 1B).

To quantify the differences in clearance pathway of ICG with or without PEG45 conjugation, the feces and urine within 24 hours from the mice with intravenous injection of ICG and ICG-PEG45, respectively, was collected. As shown in FIG. 1C, 85.3% ID (percentage of injection dose) of free ICG was found in the feces, which is 29.8 times higher than ICG-PEG45 (2.9% ID) while free ICG was undetectable in the urine and 92.9% ID of ICG-PEG45 was found in urine within 24 h post injection. Combing in vivo imaging and ex vivo clearance studies indicates that the conjugation of PEG45 nearly completely switched the elimination pathway of ICG from the hepatic clearance to the renal clearance.

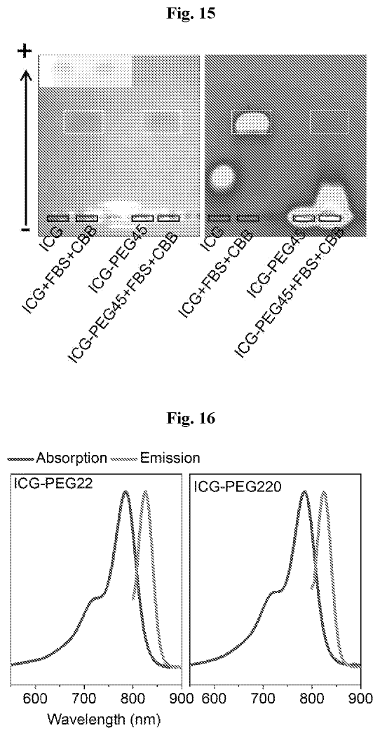

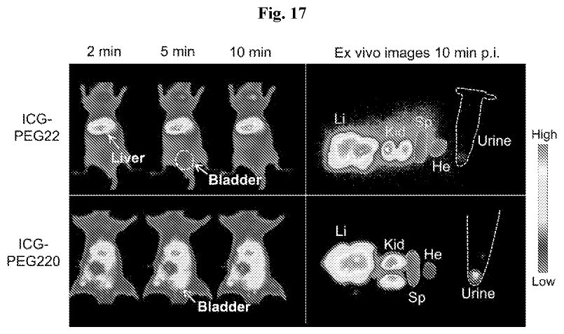

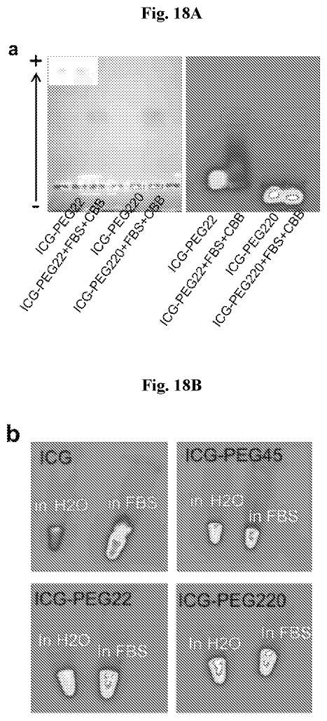

In addition to PEG45 (MW, 2100 Da), PEG22 (MW, 1100 Da) and PEG220 (MW, 10100 Da) were also conjugated to ICG, respectively. ICG-PEG22 and ICG-PEG220 retained the same absorption and photoluminescence properties as free ICG (FIG. 16) and allowed us to conduct fluorescence imaging of in vivo transport and ex vivo tissue distribution (FIG. 17). By quantifying ICG amount in feces and urine, two unique MW-dependent scaling laws in the hepatobiliary and renal clearance (FIG. 1D) was discovered. First, the amount of ICG in feces decreased with the increase of PEG molecular weight, which strongly correlated with their MW-dependent resistance to serum proteins (FIGS. 18A, 18B, and 18C). Second, the renal clearance efficiencies of ICG-PEG22 (28.9% ID) and ICG-PEG220 (44.9% ID) were both lower than that of ICG-PEG45, clearly showing the usefulness of PEG45 in tailoring the renal clearance of ICG.

### Example 2. Renal Tubular Secretion of ICG-PEG45

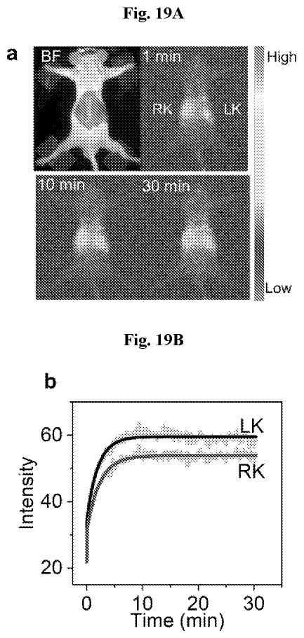

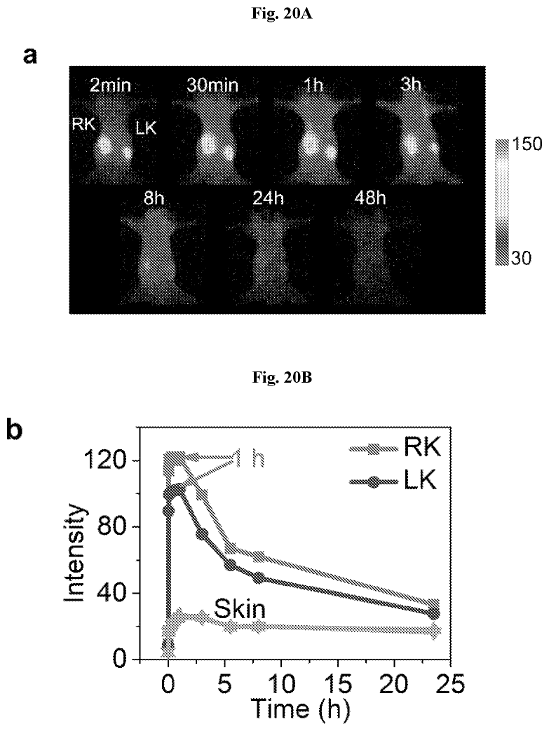

To understand how ICG-PEG45 is eliminated through the kidneys, we first used in vivo fluorescence imaging to noninvasively monitor the kidney clearance kinetics of ICG-PEG45. Interestingly, the fluorescent signal of ICG-PEG45 in the kidneys rapidly reached its maximum at ˜5 min post intravenous injection (p.i.) and remained a plateau within 30 min time period (FIG. 1E). This phenomenon was distinct to the observation of glomerular filtrated PEGylated organic dyes, such as IRDye800CW-PEG45 (800CW-PEG45), of which fluorescent signal from the kidneys reached the peak at ˜2 min p.i., followed by a rapid decay within 30 min (dot line in FIG. 1E). This plateau of ICG-PEG45 signal in the kidneys was still observed within 30 min p.i. even when the injection dose was reduced by 10 times (FIGS. 19A and 19B), suggesting that the elimination of ICG-PEG45 rapidly reached equilibrium in an early phase and the elimination kinetics was actively controlled by the kidney compartments. The plateau of ICG-PEG45 in the kidneys gradually disappeared after one hour and was followed by a descending phase (FIGS. 20A and 20B), consistent with that ICG-PEG45 was eventually eliminated through the kidneys into the urine.

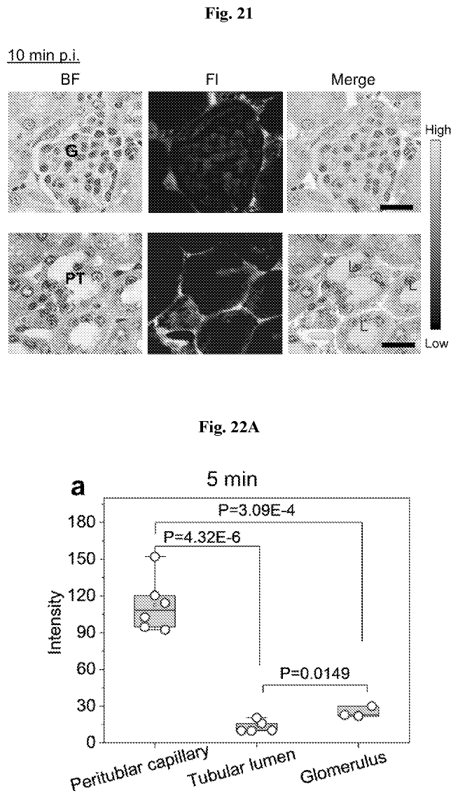

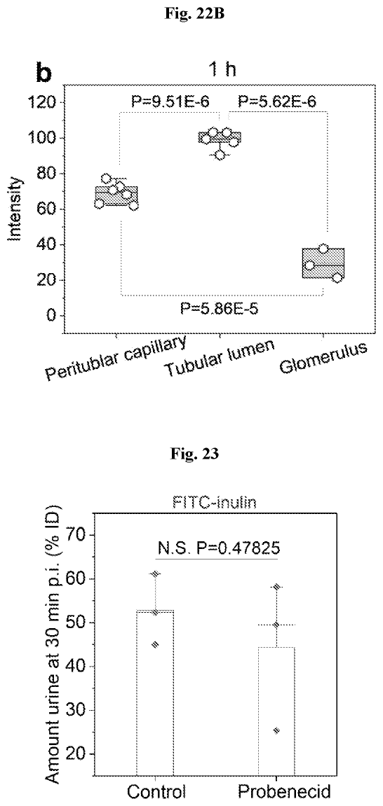

To unravel the origin of the unique time-dependent kidney fluorescence curve of ICG-PEG45, the kidneys from the mice intravenously injected with ICG-PEG45 at 5 min, 10 min and 1 h p.i. were harvested and then the distribution in the kidneys with fluorescence microscopy imaging was studied. At 5 min (FIG. 1F, right) and 10 min (FIG. 21) p.i., the fluorescence signals of ICG-PEG45 dominantly located in the peritubular capillaries, ˜4.5 times and ˜8.5 times higher than those of glomeruli and proximal tubules, respectively (FIG. 22A). In contrast, 800CW-PEG45 (FIG. 1F left) mainly located in the glomeruli and luminal side of proximal tubules at 5 min due to rapid glomerular filtration and its rapid renal clearance resulted in a very weak signal from the kidneys at 1 h p.i. Distinctly, at 1 h p.i., the peritubular space's signal of ICG-luminal side of the proximal renal tubules became much more distinguishable (FIG. 1F and FIG. 22B). The distinct kidney distribution of ICG-PEG45 from 800CW-PEG45 suggested different kidney elimination pathways and ICG-PEG45 could be directly cleared into urine through transportation from the peritubular capillary (PTC) into the tubularinterstitum (TI) and finally reached the proximal tubular lumen, the process of renal tubular secretion.

To further confirm the mechanism of active renal tubular secretion of ICG-PEG45 at the molecular level, the mice was treated with probenecid, an organic anion transporter inhibitor, to inhibit the renal basolateral uptake process without alteration of glomerular filtration (FIG. 23). As control, 800CW-PEG45 was also investigated under same probenecid treatment. As shown in FIG. 1G, the administration of probenecid significantly reduced the renal clearance efficiency of ICG-PEG45 at 30 min p.i. but did not influence the clearance of 800CW-PEG45, indicating the involvement of organic anion transporter-dependent active tubular secretion in the kidney transport of ICG-PEG45. The differences in the kidney elimination pathways between 800CW-PEG45 and ICG-PEG45 are closely related to the inherent properties of the parent molecules since free 800CW has no specific interactions with the body and was rapidly filtered through the glomeruli, whereas free ICG is known to bind to a variety of organic anion transporters. The observed renal tubular secretion of ICG after conjugation of PEG45 suggested that PEG45 enhanced the affinity of ICG to organic anion transporters on the basolateral side of proximal tubular cell (FIG. 1H) while reducing its affinity to the transporters involved in the hepatobiliary clearance.

### Example 3. Hyperfluorescent Imaging of Primary Kidney Cancers With ICG-PEG45

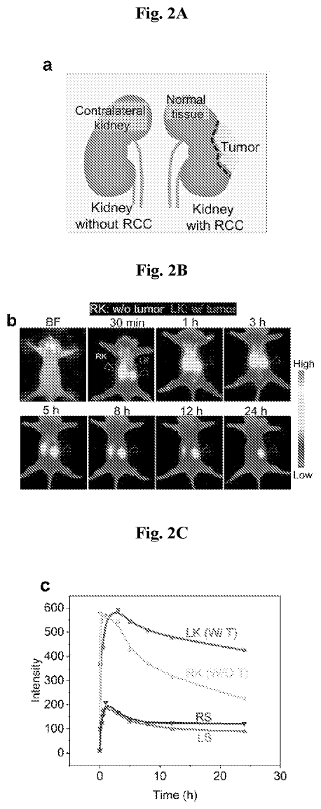

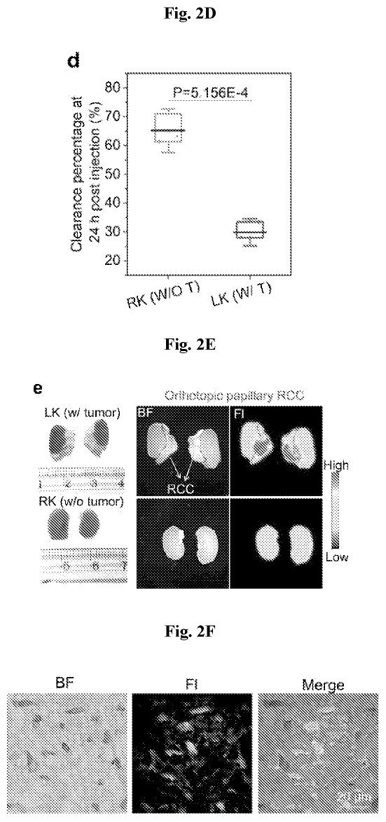

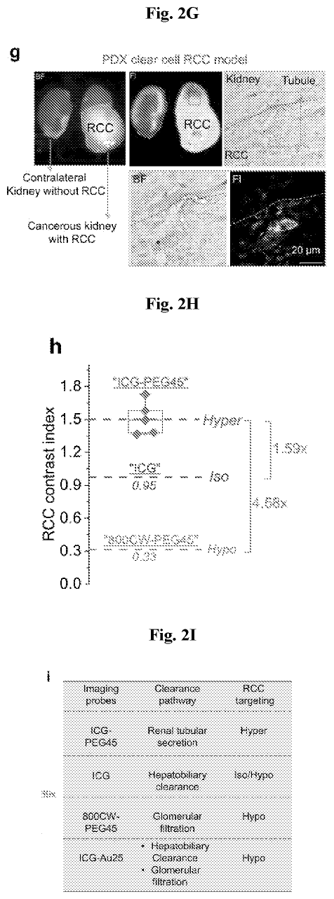

The novel renal tubular secretion pathway of ICG-PEG45 made it possible to investigate whether the renal cell carcinoma, originating from the renal tubules, can be targeted by the molecules with strong interaction with proximal tubules. An orthotopic xenograft model of papillary RCC (pRCC) was first established, which is one type of RCC that is difficult to be targeted by both passive targeting agent (such as ICG) and active targeting agent (such as ¹¹¹In-DOTA-girentuximab-IRDye800CW) due to its low expression of carbonic anhydrase IX (CAIX). The papillary RCC 529B cells (luciferase expressed) were surgically implanted into subcapsular space of the left kidney of mice and the right kidney was kept normal for renal function (FIG. 2A). When the RCC tumor developed to a size that can be reliably detected in the left kidney by bioluminescent imaging, ICG-PEG45 was intravenously injected into the mice and noninvasive in vivo fluorescence imaging was then conducted. As shown in FIGS. 2B and 2C, the fluorescence intensity observed from the contralateral right kidney initially (within 1-5 hours) was higher than that from left kidney with pRCC because ICG-PEG45 rapidly transported through the normally functionalized right kidney. As ICG-PEG45 was gradually eliminated through the right normal kidney, the fluorescence intensity observed from the left kidney with pRCC became higher than the contralateral one over time. The clearance percentage of the peak intensity of kidneys at 24 h (defined as [peak value-intensity at 24 h]/peak value]×100%) was also quantified and it was found that the value of left pRCC kidney is 29.86±3.12%, which is 2.18 times lower than that of contralateral kidney (65.15±5.09%) (FIG. 2D), suggesting the long retention of ICG-PEG45 in the pRCC kidney in comparison with normal kidney. Ex vivo fluorescence imaging of these two kidneys at 24 h post injection (FIG. 2E) further confirmed that the prolonged retention of ICG-PEG45 in the pRCC kidney specifically occurred in tumor regions: the fluorescence intensity of ICG-PEG45 in the cancerous kidney tissues is higher than that in the normal contralateral kidney, clearly indicating that ICG-PEG45 was capable of hyperfluorescently lighting up pRCC cancerous tissue and also differentiating the tumor-to-normal tissue borders. By further localizing the distribution of ICG-PEG45 in the pRCC tissue using fluorescence microscope (FIG. 2F), it was observed that ICG-PEG45 was taken up by kidney cancer cells, clearly indicating that ICG-PEG45 can selectively target kidney cancer cells. Not limited to pRCCs, ICG-PEG45 also hyperfluorescently lighted up clear cell RCCs (ccRCCs) in the patient-derived xenograft (PDX) model and successfully differentiated the tumor-to-normal tissue borders (FIG. 2G), indicating the generalizability of ICG-PEG45 in detection of multiple types of RCCs.

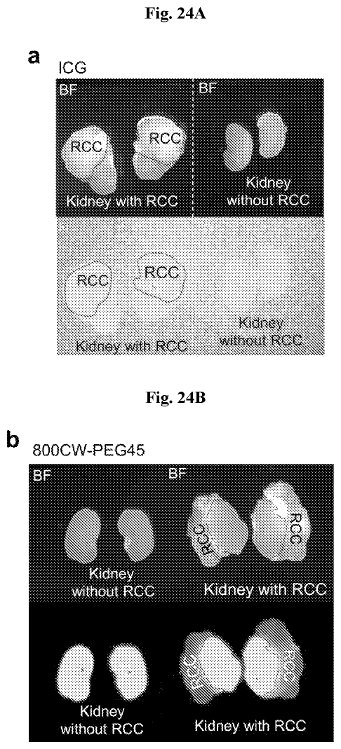

As control, the kidney cancer targeting of free ICG (cleared through the hepatobiliary clearance) and 800CW-PEG45 (cleared through the glomerular filtration) was also investigated, which both cannot reach the basolateral side of proximal tubules. As shown in FIGS. 24A and 24B, both of them failed to hyperfluorescently light up RCC in the kidneys and the contrast indexes of the tumor regions were 0.95 and 0.33 (hypofluorescent) for ICG and 800CW-PEG45, respectively, which is 1.59 times and 4.58 times lower than that of ICG-PEG45 (FIG. 2H). Moreover, the ICG was also conjugated onto renal clearable glutathione coated Au25 clusters to enhance its glomerular filtration in the kidneys but we found that it still failed to selectively target primary kidney cancers over normal kidney tissues (FIG. 25). This further confirms that kidney cancer targeting of ICG is strongly dependent of its elimination pathway in the kidneys (FIG. 2I) and the renal tubular secretion pathway allows ICG to target cancerous tubule cells through the basolateral side of proximal tubules.

### Example 4. Distinct Efflux Transport Kinetics of ICG-PEG45 in Normal and Cancerous Kidney Cells

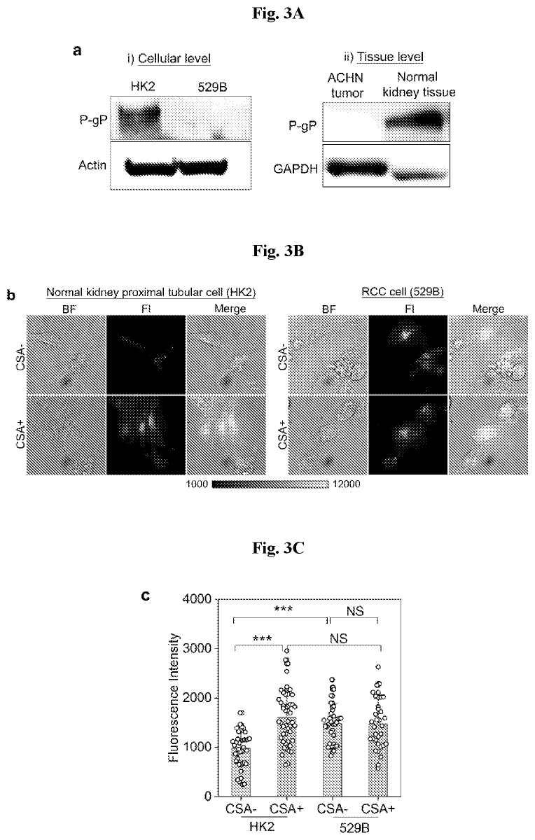

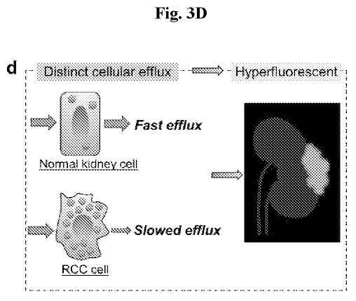

Based on the mechanism of renal tubular secretion, the cellular efflux of molecules from the proximal tubular cells into proximal tubular lumen is dictated by the efflux transporters located on the apical membrane of the proximal tubular cells, which behave as a pump to eliminate exogenous substances and dictate the intracellular drug accumulation. P-glycoprotein (P-gP) efflux transporter is well known to involve in transport of many organic molecules in the renal tubular secretion. By quantifying P-gP expression level on normal proximal tubular cells (HK2), papillary renal cancer cells (529B) as well as its expression level on normal kidney tissue and papillary renal cancerous tissue (ACHN tumor) with western blotting (FIG. 3A), we found that the expression of P-gP efflux transporter in renal cancerous tissue at both cellular and tissue level was significantly lower than that in normal proximal tubular cell and normal kidney tissues, consistent with reported literature. Thus, we hypothesized that the long retention of ICG-PEG45 in kidney cancerous tissue might be closely related to their low expression of P-gP efflux transporter.

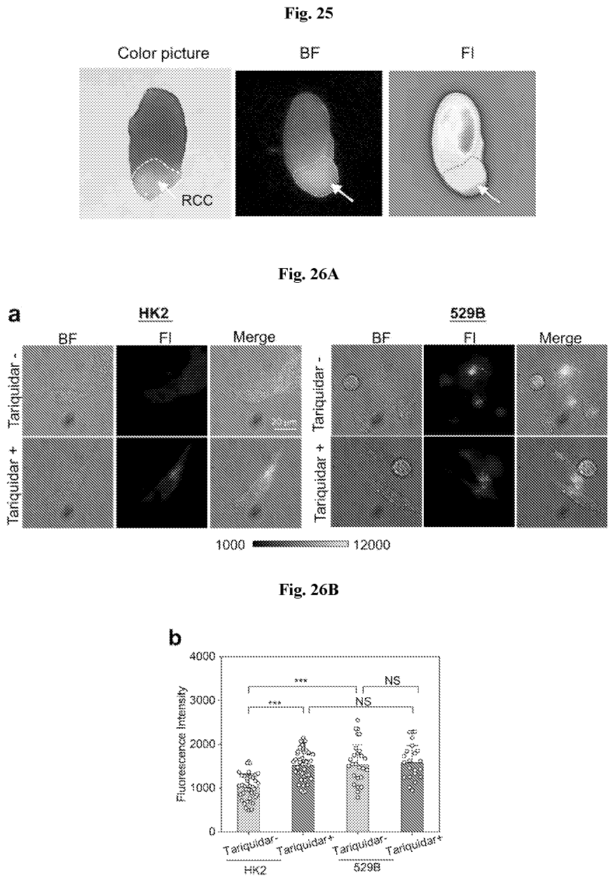

To validate our hypothesis and unravel the selective targeting of ICG-PEG45 to kidney cancers at the molecular level, we investigated in vitro cellular retention of ICG-PEG45 in normal human proximal tubular cell (HK2) and renal cancer cell (529B, papillary RCC cell line) before and after P-gP inhibitor treatment. After being incubated with ICG-PEG45 for 1 h at 37° C., both normal and cancer cells were washed with cold HEPES buffer to remove free ICG-PEG45 in the medium and the fluorescence intensities of both cells were quantified using a fluorescent microscope. As shown in FIG. 3B, ICG-PEG45 was efficiently taken up by both cell lines; but the average emission intensity of papillary RCC cells was ˜1.52 times higher than that of HK2 cells (FIG. 3C). Once we treated cells with cyclosporin A (CSA), an inhibitor of P-gP (FIGS. 3B and 3C), we found that the treatment of CSA increased the intensity of ICG-PEG45 in normal HK2 cells but did not significantly influence the intensity of ICG-PEG45 in the RCC cell line (529B), confirming that the P-gP efflux transporter was indeed involved in the secretion of ICG-PEG45 from normal proximal tubular cells. In addition, this finding also suggests that the efflux of ICG-PEG45 in renal cancer cells was compromised compared to that in the normal kidney tubular cells due to lacking P-gP efflux transporter. Moreover, when efflux was inhibited by CSA treatment, normal kidney tubular cell HK2 and 529B kidney cancerous cell showed no significant difference in fluorescence intensity, indicating that uptake efficiencies of ICG-PEG45 by normal and cancerous kidney cells were comparable. Therefore, the observed difference in the fluorescence intensity between normal and cancerous kidney without CSA treatment is due to the difference in efflux kinetics of ICG-PEG45. To further confirm the role of P-gP transporter in mediating the intracellular accumulation of ICG-PEG45, another specific P-gP inhibitor, tariquidar, was also applied to HK2 and 529B cells under same conditions and same results as CSA treatment were observed (FIGS. 26A and 26B). These findings confirmed that the difference in the efflux kinetics of ICG-PEG45 between normal kidney and cancerous tissues, governed by P-gP efflux transporter, is responsible for its selective retention in kidney tumors (FIG. 3D). This finding not only illustrated the usefulness of ICG-PEG45 in hyperfluorescent detecting of renal cell carcinoma at the molecular level but also provided a new strategy for cancer detection and targeting by taking advantage of the inherent difference in the cellular efflux of imaging agents in normal and cancerous tissue, distinct to the conventional passive targeting mechanism as well as ligand-receptor mediated active targeting strategy that have been widely used in the cancer detection through enhancing affinity of probes to cancer receptors.

### Example 5. ICG-PEG45 Selectively Detects RCC Metastasis at High Specificity

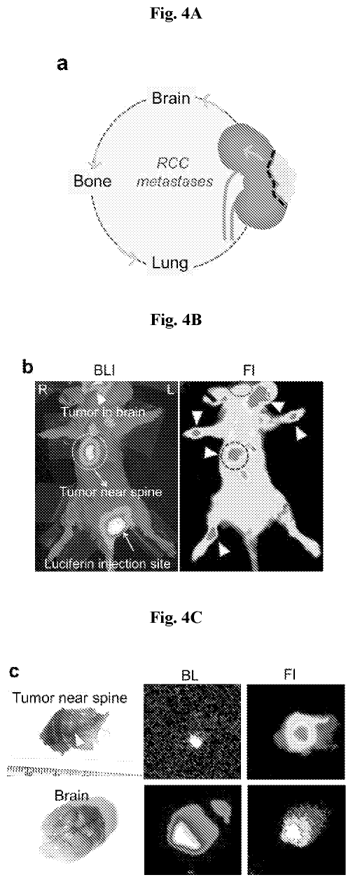

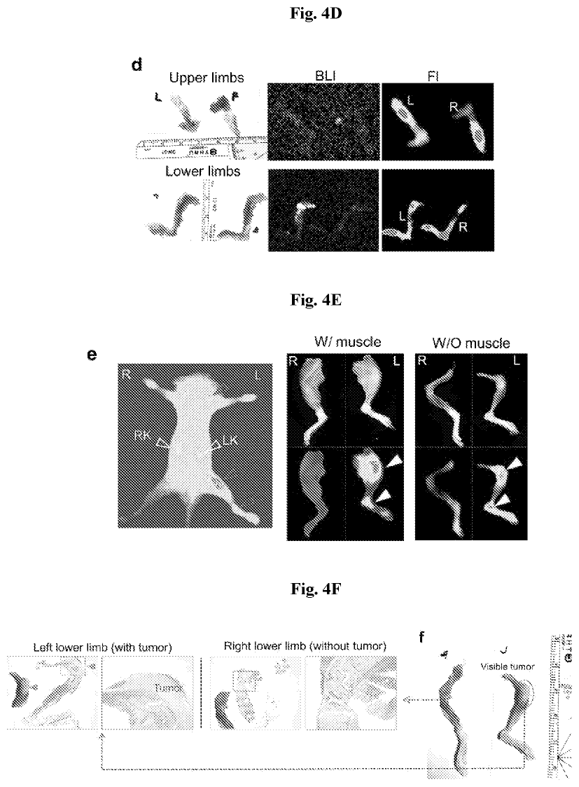

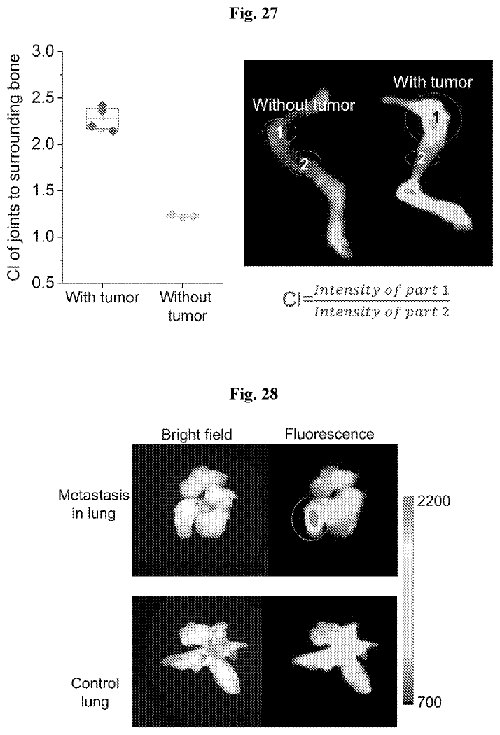

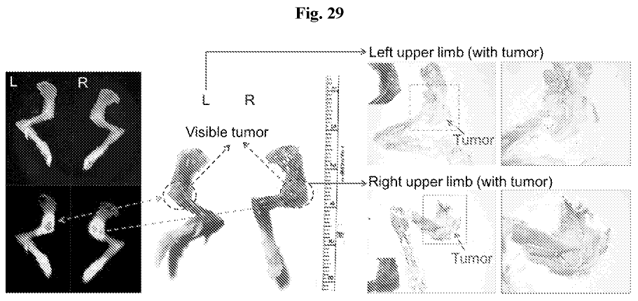

Not limited to primary RCC, we also found that ICG-PEG45 successfully detected RCC metastases in other organs such as brain, bone and lung in the mouse model (FIG. 4A). As shown in FIG. 4B, the metastatic tumors near the spine and brain were confirmed with bioluminescence and the tumor in spine can be noninvasively detected through the fluorescence of ICG-PEG45. Although the metastatic tumors in brain cannot noninvasively be observed by the fluorescence of ICG-PEG45 due to the skull, the ex vivo fluorescence imaging (FIG. 4C) clearly implied the ability of ICG-PEG45 in targeting the RCC metastatic tumors in brain. More importantly, very small tumor nodules in the bones of limbs, which cannot be detected with bioluminescence (FIG. 4D) but confirmed by H&E pathology images (FIG. 4F), were also readily detected by the fluorescence of ICG-PEG45 (FIG. 4D and FIG. 4E) with a contrast index of 2.28±0.13, ˜2 times higher than that of normal joints without tumor (FIG. 27). In addition to the RCC metastasis in brain and bone, its metastatic tumors in lungs also can be fluorescently imaged (FIG. 28). These results clearly showed that low-MW PEG45 endowed ICG the functionality in detection of RCC metastasis with positive contrast and high specificity.

### Example 6. Early Diagnosis of Renal Tubular Secretion Dysfunction and Renal Tubular Injury With ICG-PEG45.

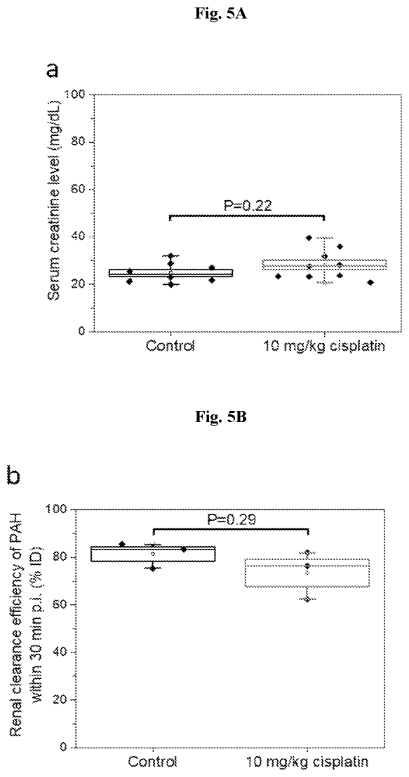

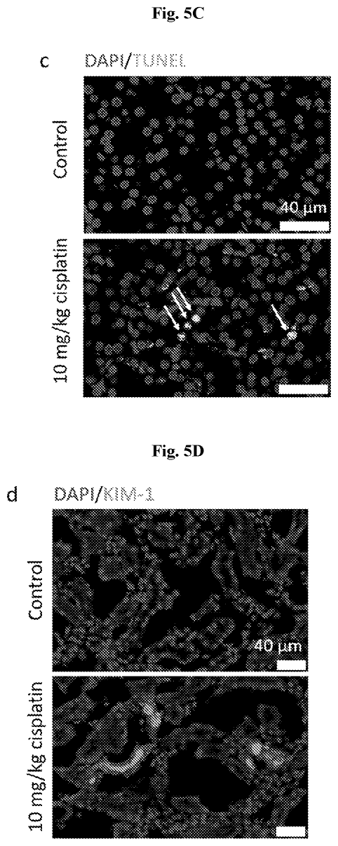

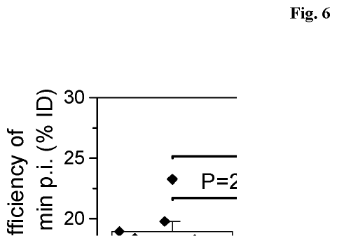

The proximal tubular dysfunction is known to significantly increase the health risk of many renal-elimination drugs; thus, the FDA regulatory guidance recommends the evaluation of tubular secretion function to personalize treatment for individual patients. However, tubular dysfunction at the early stage is difficult to detect with current small molecule-based tubular functional markers. Unlike tubular injury, which can be detected with endogenous injury markers such as KIM-1, tubular dysfunction reflected the decrease in the secretion function, which cannot be estimated with injury markers such as KIM-1. In current clinical practices, tubular secretion function is monitored with exogenous functional markers such as para-aminohippurate (PAH). By analyzing their blood and urine concentrations with “off-line” colorimetric or chromatographic methods, clinicians can quantify remaining tubular secretion function before developing personalized treatment plans. However, since PAH is small molecule and extremely efficient to be eliminated through the tubules by the transporters, renal secretion of PAH is not sensitive to early stage of tubular injury unless the tubular injury progresses into a more severe stage. Using a well-known kidney disease model, cisplatin-induced tubular injury as an example, where OAT transporters (OAT1 and OAT2 and OCT2) expression was found to be downregulated due to the injury, we found that PAH clearance and serum creatinine level changed little in the mice with a mild tubular injury induced by cisplatin at 10 mg/kg body weight even though tubular injury was validated at the tissue level using immunostaining of both tubular cell death and upregulation of kidney injury marker (KIM-1) (FIGS. 5A-5D). As shown in FIG. 6, these preliminary studies demonstrated that renal clearance of ICG-PEG45 was decreased more than 3.1 times in the tubular injury induced by cisplatin at a dose of 10 mg/kg and further decreased 9.6 times at a cisplatin dose of 20 mg/kg, clearly indicating that the ICG-PEG45 are more sensitive than PAH and Creatinine in the detection of cisplatin-induced tubular dysfunction. Thus, ICG-PEG45 could serve as either blood or urine markers for early diagnosis of tubular dysfunction and tubular injury.

### Example 7. ICG-PEG45 Serves as an Active Targeting Ligand for Efficiently Delivering Other Imaging Agents and Therapeutic Drugs to Renal Cell Carcinoma (RCC)

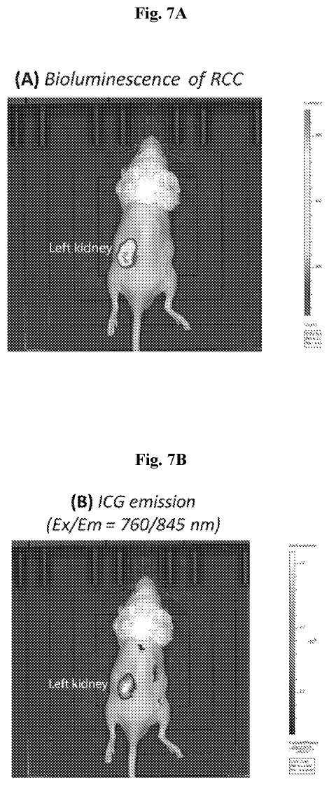

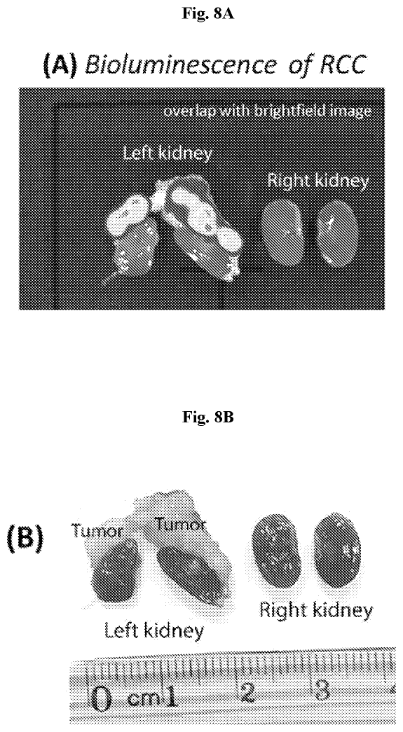

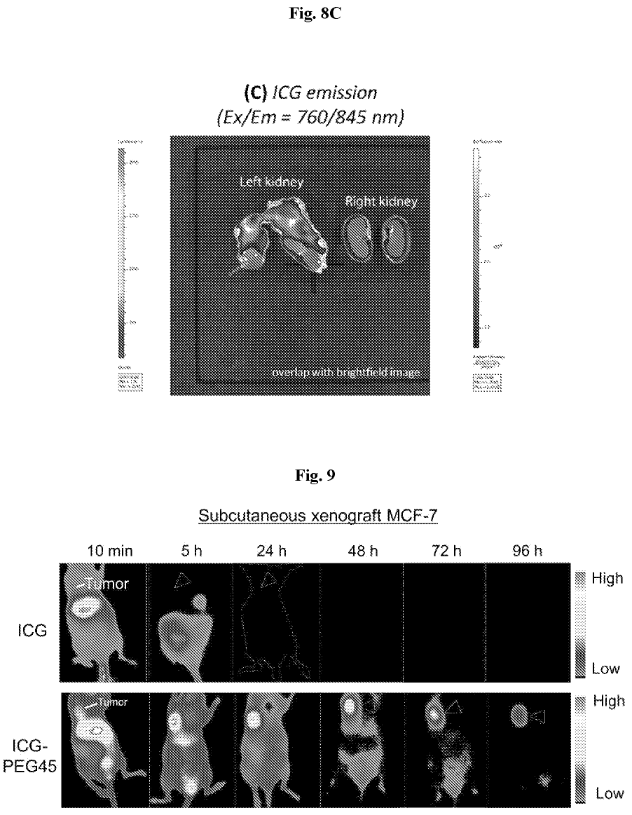

As an example, we conjugated ICG-PEG45 to 1,4,7,10-Tetraazacyclododecane-1,4,7,10-tetraacetic acid (DOTA). DOTA is a clinically used chelating agent that can form complexes with gadolinium for application as MRI contrast agent or form complexes with radioisotopes such as 64Cu and 68Ga for positron emission tomography (PET). PET has become as one of the most important imaging modalities in staging, detecting recurrence and metastasis, and monitoring treatment efficacy in most cancers. However, RCC cannot be accurately diagnosed with PET after injection of [18F]fluorodeoxyglucose (FDG), the most commonly used PET agent, mainly because physiological excretion of FDG from the kidneys reduces the contrast between malignant and normal kidney tissues. To demonstrate the RCC targeting of ICG-PEG45-DOTA, we first established orthotopic RCC xenograft mouse model. The papillary RCC cell line (ACHN) transfected with luciferase-expression vector was surgically implanted into subcapsular space of the left kidney of mice and the right kidney was kept normal for renal function. When the RCC tumor developed to a size that can be reliably detected in the left kidney by bioluminescent imaging, we intravenously injected ICG-PEG45-DOTA into the mice and then conducted noninvasive in vivo fluorescence imaging at 24 h post injection of ICG-PEG45-DOTA (200 μL, 40 μM). As shown in FIG. 7A, strong bioluminescence signal was detected on the left kidney indicated the growth of RCC. At 24 h post injection of ICG-PEG45-DOTA, near-infrared fluorescence image of the same mouse suggests that ICG-PE45-DOTA can specifically accumulate in the left kidney with RCC but can be cleared from the normal right kidney (FIG. 7B). Ex vivo imaging of these two kidneys (FIGS. 8A-8C) further confirmed that the prolonged retention of ICG-PEG45-DOTA in the left kidney specifically occurred in tumor regions. The malignant kidney tissues can be validated by bioluminescence imaging and the tumor was white (FIG. 8A and FIG. 8B). Interestingly, the fluorescence intensity of ICG-PEG45-DOTA in the malignant tissues was higher than that in normal kidney tissues in both left and right kidneys (FIG. 8C). These results clearly indicate that ICG-PEG45 can be used as an active targeting ligand to efficiently deliver other imaging agents and therapeutic drugs to RCC once ICG-PEG45 is conjugated to the agents.

### Example 8. ICG-PEG45 Selectively Targets and Detects Breast Cancer at High Specificity

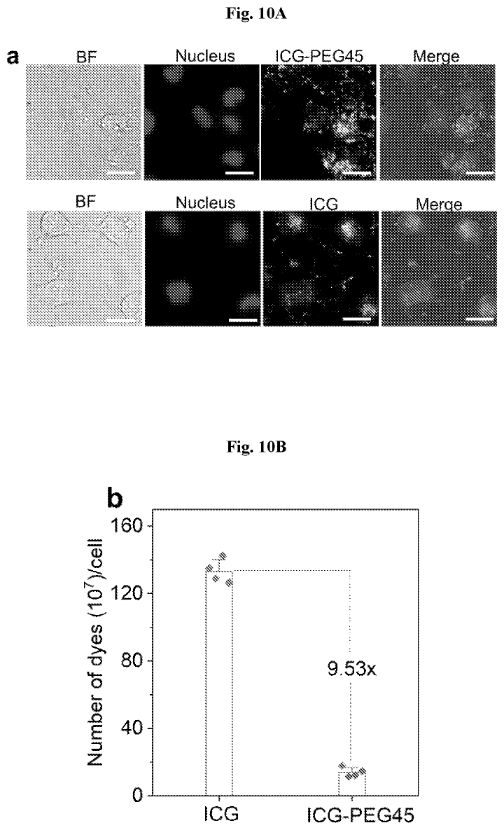

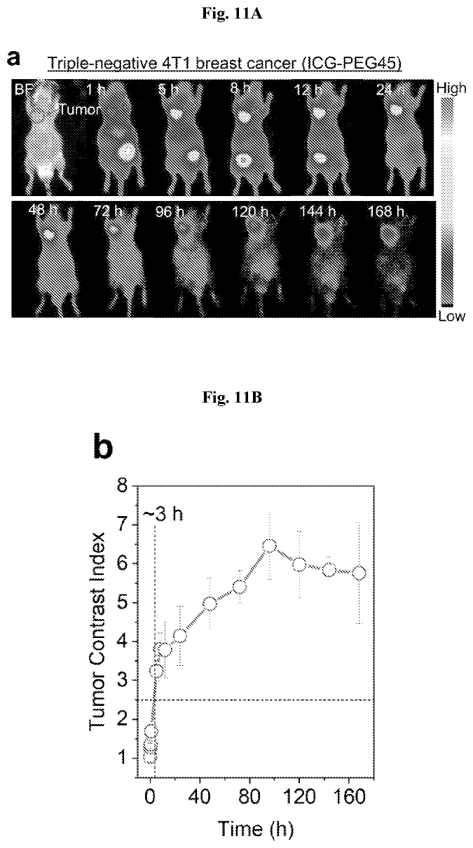

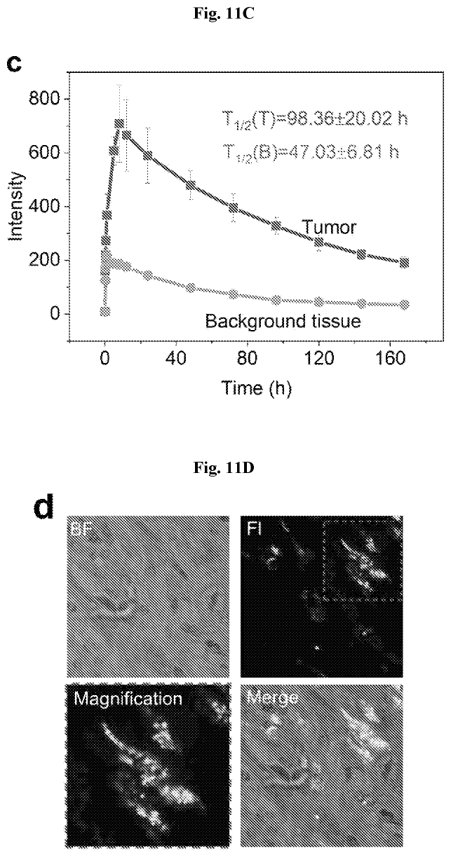

MCF-7 triple negative breast tumor could also be selectively targeted with ICG-PEG45 because OATP1A2 was overexpressed; so that selective accumulation of ICG-PEG45 in MCF-7 tumor was observed in the tumor-bearing mice. By conducting a head-to-head comparison of ICG and ICG-PEG45 in real-time imaging of MCF-7 tumors in a subcutaneous xenograft (FIG. 9), iwas found that ICG-PEG45 not only enabled the tumor visualization at a contrast index higher than 4 but also retained the high imaging contrast for at least 4 days even though the cellular uptake efficiency of ICG-PEG45 is nearly 10 times lower than that of free ICG (FIGS. 10A-10B), indicating that the tumor targeting of ICG after PEG45 conjugation was largely dictated by its in vivo transport rather than its cellular interactions.

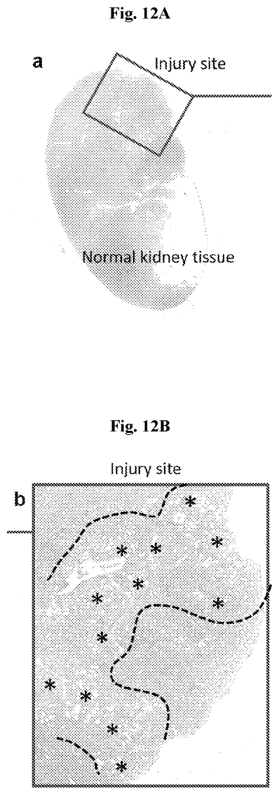

Not limited to visualization of MCF-7 tumor model, ICG-PEG45 also readily detected triple-negative 4T1 breast cancer. As shown in FIG. 11A and FIG. 11B, imaging contrast of 4T1 breast tumor can be retained above a contrast index of 6 for at least 4 days and a 2.5 contrast index for more than 6 days. Such high fluorescence contrast index of the tumor fundamentally arises from two unique reasons involving transport and interactions of ICG-PEG45 within the tumor. The first reason is that the clearance of ICG-PEG45 in background tissue is much faster than it is in tumor. As shown in FIG. 11C, the decay half-life of ICG-PEG45 in the tumor is 98.36±20.02 h, which is ˜2 times longer than that in background tissue (47.03±6.81 h). The second reason is that ICG-PEG45 was found to be readily taken up by the cells in the tumor (FIG. 11D), which slows down its clearance from the tumor microenvironment. The unique in vivo transport and interactions of ICG-PEG45 in the tumor microenvironment and background tissue are responsible for its high imaging contrast for a long period of time.

### Example 9. ICG-PEG Selectively Targets and Detects Injured Renal Tubules

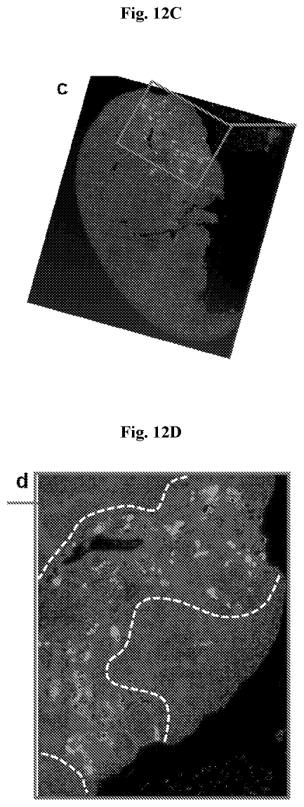

A mouse received a surgical trauma of renal cortex, which induced tubular injury. At 2 weeks after the surgery, ICG-PEG (MW:5000 Da) was intravenously injected to this mouse and the kidney was collected at 4 days post ICG-PEG injection. The kidney was fixed, processed, and embedded in paraffin. To identify the intra-kidney distribution of ICG-PEG, two consecutive sections of the kidney were stained with Hematoxylin and Eosin (H&E, for pathological analysis) and stained with DAPI (for nuclei staining and fluorescence imaging), respectively. FIG. 12A shows the entire cross section of H&E-stained kidney section. The dilated renal tubules and interstitial infiltration of immune cells (labeled by stars in FIG. 12B) were clearly observed, indicating tubular injury caused by the surgical cut. FIGS. 12C and 12D show the overlay of ICG image (displayed in red) and DAPI image (displayed in blue, nuclei staining). Interestingly, ICG-PEG (MW of PEG=5000) selectively accumulated inside of the injured renal tubular cells (FIGS. 12C and 12D), whereas the normal renal tubule cells had very weak near-infrared fluorescence.

### Example 10. IRDye 800CW-Conjugated PEG45 Fails to Detect Proximal Tubular Injury in Early Stages Before the Elevation of Blood Urea Nitrogen (BUN) and Creatinine, the Two Conventional Kidney Function Biomarkers

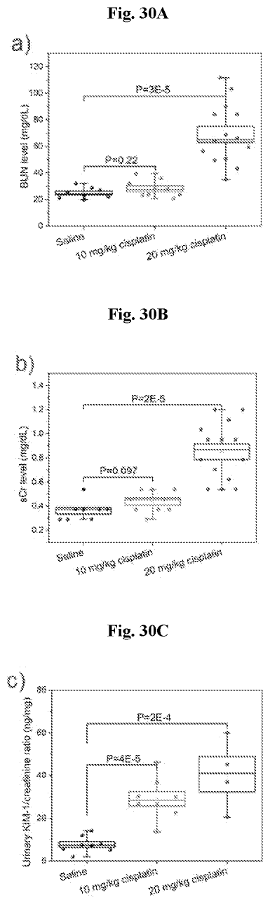

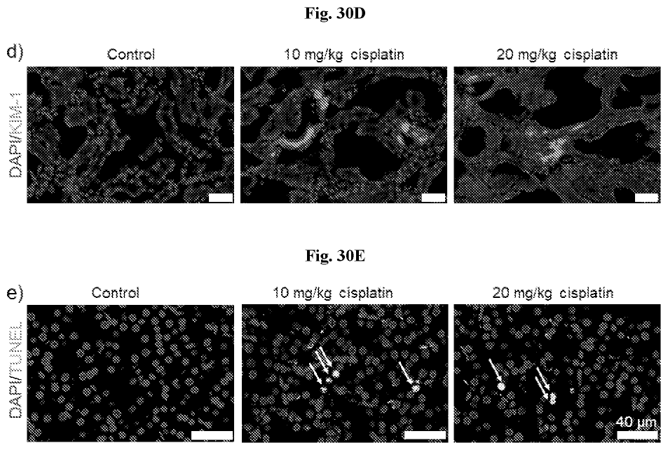

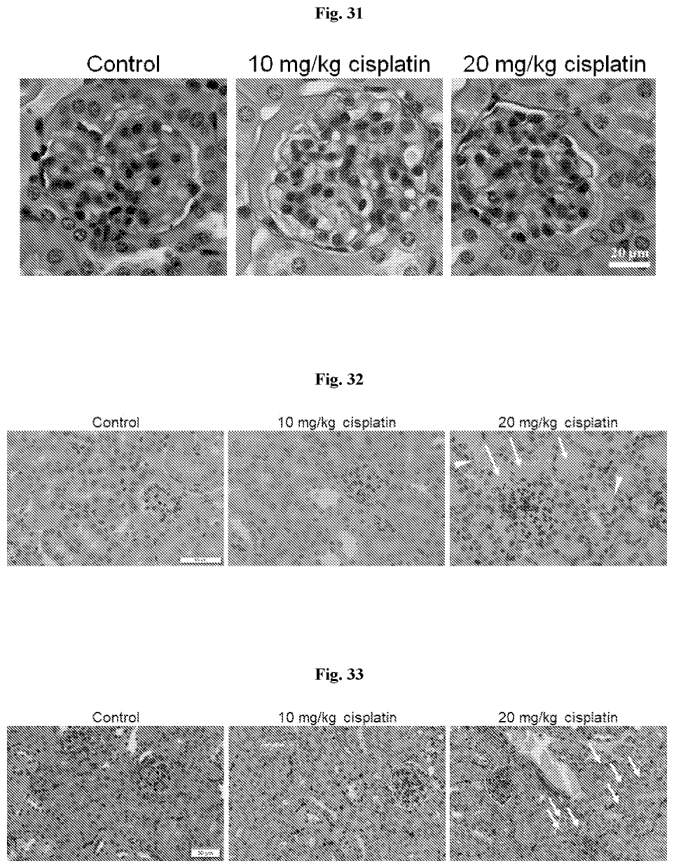

Comparative studies have shown the IRDye800CW-PEG45 was rapidly and passively filtered through the glomeruli into the cavities of the proximal tubules while ICG-PEG45 was actively secreted by organic anion transporters (OATs) and P-glycoprotein (P-gp) transporters of the proximal tubules directly from the blood stream into the cavities of proximal tubules without passing through the glomeruli in the normal kidneys. To gain the deep understanding of how the kidney transport of these two dye-labeled PEG nanoparticles in the diseased kidneys with tubular injury at a very early stage, we used cisplatin, a well-known nephrotoxic anticancer drug, to induce a very mild tubular injury but without damaging the glomeruli at a dose of 10 mg/kg body weight. As shown in FIGS. 30A and 30B, at 4 days after intraperitoneal injection of 10 mg/kg cisplatin, the levels of conventional renal function biomarkers such as blood urea nitrogen (BUN) and serum creatinine (sCr) remained comparable to those of normal mice receiving saline injection. Pathological analysis of the kidneys showed no damage to the glomeruli at 4 days after intraperitoneal injection of 10 mg/kg cisplatin (FIG. 31). On the other hand, one of the most sensitive biomarkers for tubular injury, Kidney Injury Molecule-1 (KIM-1) in the urine, indeed showed significant increase after being normalized using urinary creatinine (KIM-1/creatinine ratio; FIG. 30C). The increase of urine KIM-1/creatinine ratio was also consistent with the observation that KIM-1 protein expression was significantly increased on the proximal tubules (FIG. 30D). The elevation of KIM-1 level in both kidney tissues and urine was because tubular cell apoptosis upregulates KIM-1 expression. Additional TUNEL assays (Terminal deoxynucleotidyl transferase (TdT) dUTP Nick-End Labeling assay) also indicated apoptosis involved in the cisplatin-induced tubular injury (FIG. 30E). Once the cisplatin dose was increased to 20 mg/kg, the proximal tubules were more severely damaged (FIG. 32). Not only KIM-1/creatinine ratios and BUN and creatinine levels were further increased (FIGS. 30A-30C), protein casts induced by the dead cells were also observed (FIG. 33), which are known to cause tubular blockages.

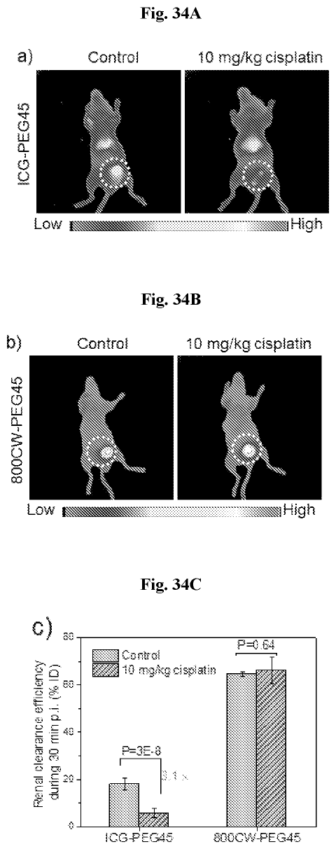

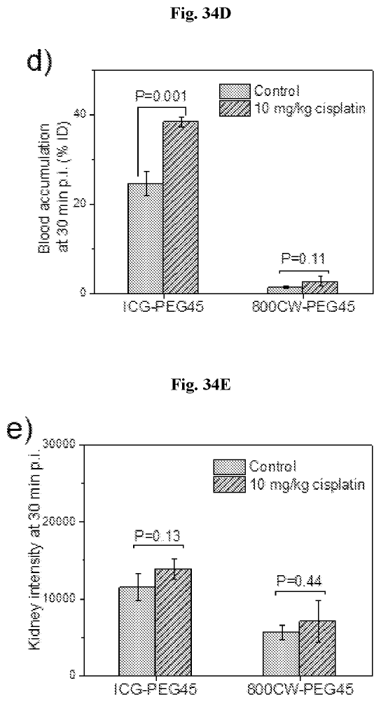

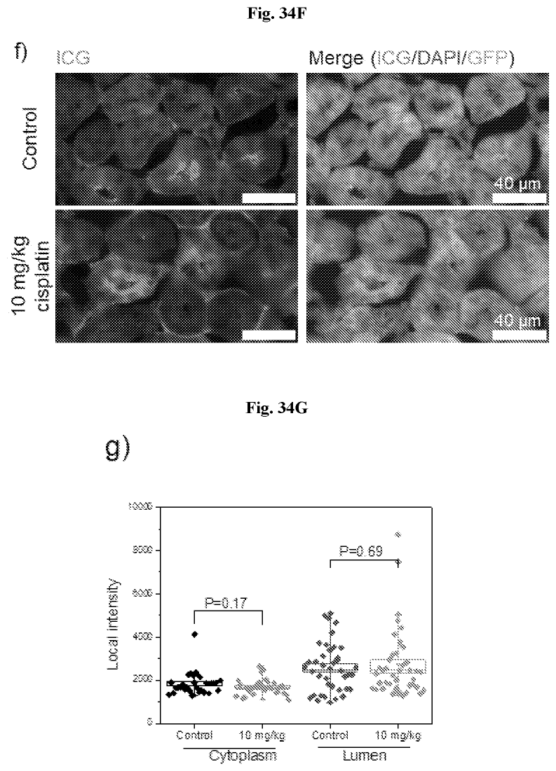

In order to unravel the potential differences in the transport of ICG-PEG45 and IRDye800CW-PEG45 in the kidneys at an early stage of tubular injury, we intravenously injected them into normal mice and the mice with tubular injury induced by cisplatin at a dose of 10 mg/kg at first. By quantifying the amount of both nano-fluorophores in the urine at 30 min post injection, we found that the renal clearance efficiency of ICG-PEG45 decreased from 18.1 ±2.5%ID to 5.8±1.9% ID (% ID=percentage of injected dose), ˜3.1 times reduction in the diseased mice compared to that in the normal ones (FIGS. 34A and 34C), while little difference in 30-min renal clearance was observed from IRDye800CW-PEG45 (64.6±0.9% ID and 66.2±5.6% ID) (FIGS. 34B and 34C). To understand the origin of the reduction in the renal clearance of ICG-PEG45, we measured the blood accumulation of ICG-PEG45 at 30 min. p.i. in the normal and cisplatin-treated mice. With an assumption that the blood volume is 7% of the mouse body weight, the accumulation of ICG-PEG45 in the blood of normal and diseased mice was quantified to be 24.6±2.6% ID and 38.4±1.0% ID, respectively, suggesting that an addition 14% ID of ICG-PEG45 was retained in the blood of mice receiving 10 mg/kg cisplatin (FIG. 34D). The finding was consistent with the decrease in the renal clearance of ICG-PEG45 from the normal mice to cisplatin-treated mice (12% ID). In addition, ex vivo kidney images of normal and diseased mice at 30 min after intravenous injection of IRDye800CW-PEG45 and ICG-PEG45, respectively, show no significant differences in the kidney fluorescence intensities (FIG. 34E). Furthermore, fluorescence imaging of the frozen tissue slides of the normal and injured kidneys collected from the mice intravenously injected with ICG-PEG45 show comparable intensities of cytoplasm and lumens suggest that ICG-PEG45 was still successfully secreted out of the tubules once it was taken up by the proximal tubular cells (FIGS. 34F-34G). Combining all the results indicates that cisplatin-induced proximal tubular injury at this very early significantly reduced the cellular uptake of ICG-PEG45 from the blood, leading to increased blood centration of ICG-PEG45 and reduced renal clearance. In contrast, glomerular-filtered IRDye800CW-PEG45 showed little differences in either its renal clearance or blood retention in mice at 4 days after intraperitoneal injection of 10 mg/kg cisplatin (FIGS. 34C-34D).

In summary, at a very early tubular injury stage induced by cisplatin at a dose of 10 mg/kg (at day 4 after intraperitoneal injection), where only minor tubular injury but no glomerular injury and elevation of blood urea nitrogen (BUN) and creatinine were observed, renal clearance of ICG-PEG45 was reduced by nearly 3.1 times while its blood retention was increased about 1.6 times compared to those in the normal mice. However, glomerular-filtered IRDye800CW-PEG45 showed little differences in either its renal clearance or blood retention at this same early stage of proximal tubular injury.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of diagnosing a disease or condition associated with abnormal expression of an influx or efflux transporter in a subject, comprising:
administering to the subject a composition comprising polyethylene glycol (PEG) conjugated to indocyanine green (ICG) (“ICG-PEG conjugate”);
determining a concentration of the ICG-PEG conjugate in a biological sample obtained from the subject;
comparing the concentration of the ICG-PEG conjugate with a reference level; and
determining that the subject has the disease or condition if the concentration of the ICG-PEG conjugate is significantly greater or lower than the reference level.

2. A method of diagnosing a disease or condition associated with abnormal expression of an influx or efflux transporter in a subject, comprising:
administering to the subject a composition comprising polyethylene glycol (PEG) conjugated to indocyanine green (ICG) (“ICG-PEG conjugate”);
measuring an intensity of a signal from the ICG-PEG conjugate in a tissue of the subject;
comparing the intensity with a reference level; and
determining that the subject has the disease or condition if the intensity is significantly greater or lower than the reference level.

3. The method of claim 2, wherein the ICG-PEG conjugate provides a contrast index of at least 1.5.

4. A method of monitoring kidney secretion function of a subject, comprising:
administering to the subject a composition comprising polyethylene glycol (PEG) conjugated to indocyanine green (ICG) (“ICG-PEG conjugate”);
determining a first concentration of the ICG-PEG conjugate in a first biological sample obtained from the subject at a first time point;
determining a second concentration of the ICG-PEG conjugate in a second biological sample obtained from the subject at a second time point, wherein the second time point is after the first time point;
determining renal clearance kinetics based on the first concentration and the second concentration; and
optionally comparing the renal clearance kinetics with a reference level.

5. A method of monitoring kidney secretion function of a subject, comprising:
administering to the subject a composition comprising polyethylene glycol (PEG) conjugated to indocyanine green (ICG) (“ICG-PEG conjugate”);
measuring, at a first time point, a first intensity of a signal from the ICG-PEG conjugate in a tissue of the subject; and
measuring, at a second time point, a second intensity of a signal from the ICG-PEG conjugate in the tissue of the subject.

6. A method of treating a disease or condition associated with abnormal expression of an influx or efflux transporter in a subject in need thereof, comprising administering to the subject a composition comprising polyethylene glycol (PEG) conjugated to indocyanine green (ICG) (“ICG-PEG conjugate”).

7. A method of detecting a liver disease in a subject, comprising:
administering to the subject a composition comprising polyethylene glycol (PEG) conjugated to indocyanine green (ICG) (“ICG-PEG conjugate”), wherein the PEG has a molecular weight of at least 100 Da to less than 2 kDa;
determining a concentration of the ICG-PEG conjugate in a urine sample obtained from the subject;
comparing the concentration of the ICG-PEG conjugate with a reference level; and
determining that the subject has the liver disease when the concentration of the ICG-PEG conjugate is significantly less than the reference level.

8. A method of measuring an expression level of an influx or efflux transporter in a subject, comprising:
administering to the subject a composition comprising polyethylene glycol (PEG) conjugated to indocyanine green (ICG) (“ICG-PEG conjugate”);
determining a concentration of the ICG-PEG conjugate in a biological sample obtained from the subject; and
determining the expression level of the influx or efflux transporter based on the concentration of the ICG-PEG conjugate.

9. A method of measuring an expression level of an influx or efflux transporter in a subject, comprising:
administering to the subject a composition comprising polyethylene glycol (PEG) conjugated to indocyanine green (ICG) (“ICG-PEG conjugate”);
measuring an intensity of a signal from the ICG-PEG conjugate in a tissue of the subject; and
determining the expression level of the influx or efflux transporter based on the intensity.

10. The method of claim 1, wherein the subject has upregulated or downregulated expression of P-glycoprotein (P-gP), multidrug-resistant protein 2 (MRP2), MRP4, an organic cation transporter (OCT), an organic anion transporter (OAT), an organic anion-transporting polypeptide (OATP), breast cancer resistance protein (BCRP), or organic anion transporter 4 (OAT4), equilibrative nucleoside transporter 1 (ENT1), ENT2, organic solute transporter α (OSTα), or OSTβ.

11. The method of claim 1, wherein the disease or condition is renal tubular secretion dysfunction or renal tubular injury.

12. The method of claim 11, wherein the renal tubular secretion dysfunction or renal tubular injury is proximal renal tubular secretion dysfunction or proximal renal tubular injury.

13. The method of claim 11, wherein the renal tubular secretion dysfunction or renal tubular injury is associated with a kidney disease or condition selected from acute kidney injury, chronic kidney injury, kidney cancer, lupus nephritis, diabetes-induced kidney injury, polycystic kidney disease, sepsis, kidney inflammation, kidney transplant rejection, and kidney dysfunction or kidney injury caused by diseases in other tissues and organs such as cancer and liver diseases.

14. The method of claim 1, wherein the disease or condition is kidney cancer, breast cancer, liver cancer, ovarian cancer, bladder cancer, prostate cancer, lung cancer, pancreatic cancer, bone cancer, or colon cancer.

15. The method of claim 14, wherein the kidney cancer is renal cell carcinoma or renal oncocytoma.

16. The method of claim 15, wherein the kidney cancer is renal cell carcinoma.

17. The method of claim 16, wherein the renal cell carcinoma is clear cell renal cell carcinoma (ccRCC), or papillary RCC (pRCC).

18. The method of claim 14, wherein the disease or condition is breast cancer, and wherein the breast cancer is triple negative breast cancer.

19. The method of claim 1, wherein the biological sample is a blood or urine sample.

20. The method of claim 1, wherein the biological sample is a urine sample.

21. The method of claim 1, wherein the ICG-PEG conjugate is of formula I: 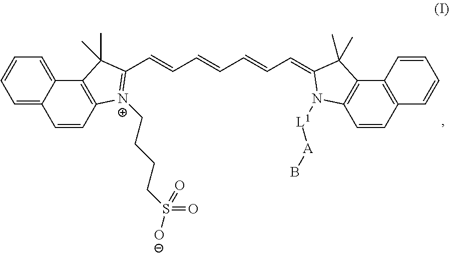
or a pharmaceutically acceptable salt thereof, wherein:
L¹ is independently optionally substituted alkylene, haloalkylene, alkenylene or alkynylene;
A is independently —C(O)NH(CH₂CH₂O)ₙ—, —C(O)O(CH₂CH₂O)ₙ—, —C(O)S(CH₂CH₂O)ₙ—, —NHC(O)CH₂O(CH₂CH₂O)ₙ—, —OC(O)CH₂O(CH₂CH₂O)ₙ—, or —SC(O)CH₂O(CH₂CH₂O)ₙ—, wherein the (CH₂CH₂O)— end is connected to B;
n is an integer selected from about 10 to about 1000; and
B is independently H, or optionally substituted alkyl.

22. The method of claim 21, wherein L¹ is unsubstituted C₁₋₆ alkylene or C₁₋₆ haloalkylene.

23. The method of claim 21, wherein B is H or unsubstituted C₁₋₆ alkyl.

24. The method of claim 21, wherein B is C₁₋₆ alkyl substituted with one or more —OH, —NH₂, —SH, or —COOH.

25. The method of claim 23, wherein B is —CH₂CH₂OH, —CH₂CH₂NH₂, —CH₂CH₂SH, —CH₂CH₂C(O)OH, or —CH₂C(O)OH.

26. The method of claim 21, wherein the ICG-PEG conjugate is of the formula 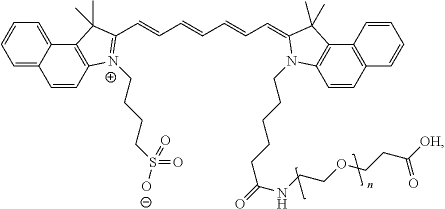
or a pharmaceutically acceptable salt thereof.

27. The method of claim 21, wherein n is an integer selected from about 22 to about 220.

28. The method of claim 21, wherein n is about 45.

29. The method of claim 1, wherein the conjugate is in the form of nanoparticles.

30. The method of claim 29, wherein the nanoparticles have an average diameter of about 0.5 nm to about 12 nm.

31. The method of claim 1, wherein the composition is administered intravenously, intraperitoneally, subcutaneously, or intraarterially.

32. The method of claim 1, wherein the ICG-PEG conjugate is further conjugated to an imaging agent, biochemical activatable agent, or a therapeutic agent.

33. A composition comprising indocyanine (ICG), polyethylene glycol (PEG), and a secondary moiety, wherein ICG and the secondary moiety are each independently conjugated to PEG.

34. The composition of claim 33, wherein the secondary moiety is an imaging agent, biochemical activatable agent, or a therapeutic agent.
